(19) United States
(12) Patent Application Publication
(10) Pub. No.: US 2024/0172990 A1
(43) Pub. Date: May 30, 2024

(54) PREPARTUM AND POSTPARTUM MONITORING AND RELATED RECOMMENDED MEDICAL TREATMENTS

(71) Applicant: NAVIGATE MATERNITY INC., Leo, IN (US)

(72) Inventor: Ariana Mcgee, Leo, IN (US)

(21) Appl. No.: 18/525,548

(22) Filed: Nov. 30, 2023

Related U.S. Application Data
(60) Provisional application No. 63/429,066, filed on Nov. 30, 2022.

(51) Int. Cl. A61B 5/00; A61B 5/0205; A61B 5/16; G16H 10/20

(52) U.S. Cl. CPC A61B 5/4343; A61B 5/0205; A61B 5/165; A61B 5/486; A61B 5/7267; A61B 5/746; G16H 10/20; A61B 5/021

(57) ABSTRACT

The following relates generally to perinatal monitoring of a patient, and recommending treatments and/or clinician appointments for the perinatal patient. In some embodiments, a computing device of a perinatal patient sends, to a healthcare computing device and/or a clinician computing device: (i) blood pressure data and/or heart rate data of the perinatal patient, (ii) answers to depression survey questions, and/or (iii) answers to social determinants of health score survey questions. In some embodiments, a display device of the clinician computing device displays: (i) the blood pressure data and/or heart rate data of the perinatal patient, (ii) a depression score of the perinatal patient, and (iii) a social determinant of health score of the perinatal patient. Some embodiments also display graphical trends in the (i) blood pressure data and/or heart rate data, (ii) depression scores, and (iii) social determinant of health scores.

### CROSS-REFERENCE TO RELATED APPLICATIONS

The present application claims priority to U.S. Provisional App. No. 63/429,066, entitled “PREPARTUM AND POSTPARTUM MONITORING AND RELATED RECOMMENDED MEDICAL TREATMENTS,” and filed Nov. 30, 2022, the entire disclosure of with is incorporated by reference herein.

### BACKGROUND

During the perinatal experience, prenatal and postpartum (i.e., during and after pregnancy), a patient may experience significant physical and physiological medical conditions. For example, postpartum depression and/or preeclampsia often go undetected or untreated, harming many new mothers. Sadly, failure to detect and intervene on these medical conditions lead to significantly higher morbidity and mortality rates among certain racial minority populations.

The systems and methods disclosed herein provide solutions to these problems and others.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one aspect, a computer-implemented method for monitoring a perinatal patient from diagnosis through twelve months postpartum to a childbirth event via real time patient data and alerts. The method may include: receiving, via one or more processors, biometric data of a patient (e.g., including blood pressure data, heart rate data, weight data, glucose monitoring data, etc.); receiving, via the one or more processors, from a patient user device corresponding to the patient, a plurality of answers corresponding to respective depression survey questions; determining, via the one or more processors, a depression score of the patient based on the received plurality of answers corresponding to respective depression survey questions; receiving, via the one or more processors, from the patient user device, a plurality of answers corresponding to social determinants of health survey questions; determining, via the one or more processors, a social determinants of health score of the patient based on the received plurality of answers corresponding to respective social determinants of health survey questions; and presenting, via the one or more processors, to a clinician: (i) the biometric data of the patient, (ii) the depression score of the patient, and (iii) the social determinants of health score of the patient. The method may include additional, fewer, or alternate actions, including those discussed elsewhere herein.

### BRIEF DESCRIPTION OF THE DRAWINGS

FIG. 1 illustrates an example perinatal monitoring and recommendation architecture.

FIG. 2 illustrates an example display allowing the postpartum patient to login.

FIG. 3 illustrates an example display allowing the postpartum patient to enter a personal identification number (PIN) as part of logging in.

FIG. 4 illustrates an example display where no patient data has been detected.

FIG. 5 illustrates an example display populated with the postpartum patient's data, such as blood pressure data and heart rate data. The example display also includes scroll buttons.

FIG. 6 illustrates an example display displaying an example social health score.

FIG. 7 illustrates an example display displaying an example depression score.

FIG. 8 illustrates an example display displaying example social health survey questions.

FIG. 9 illustrates an example display displaying aspects of flagged responses of an example social health survey.

FIG. 10 illustrates an example display displaying example responses of an example social health survey.

FIG. 11 illustrates an example display displaying example past scores of social health surveys.

FIG. 12 illustrates an example display displaying an example beginning to an example social health survey.

FIG. 13 illustrates an example display displaying an example social health survey question.

FIG. 14 illustrates an example display displaying an example social health survey question with an answer filled in.

FIG. 15 illustrates an example display indicating that a social health survey is complete.

FIG. 16 illustrates an example display including an alert that the perinatal patient may need additional help following a social health survey.

FIG. 17 illustrates an example display displaying a beginning to an example depression survey.

FIG. 18 illustrates an example display displaying an example depression survey question with an answer filled in.

FIG. 19 illustrates an example display displaying an example depression survey question without an answer filled in.

FIG. 20 illustrates an example display displaying another example depression survey question without an answer filled in.

FIG. 21 illustrates an example display displaying another example depression survey question without an answer filled in.

FIG. 22 illustrates an example display indicating that a depression survey is complete.

FIG. 23 illustrates an example display including an alert that the perinatal patient may need additional help following a depression survey.

FIG. 24 illustrates an example display displaying a selection of text message conversations.

FIG. 25 illustrates an example display displaying a particular text message conversation.

FIG. 26 illustrates an example display displaying a list of tasks.

FIG. 27 illustrates an example display displaying depression survey and depression score information.

FIG. 28 illustrates an example display where all of blood pressure data, heart rate data, depression score data, social health score data, and a text message conversation are all displayed.

FIG. 29 illustrates an example display including a list of electronic medical devices.

FIG. 30 illustrates an example display allowing a clinician to search for a perinatal patient.

FIG. 31 illustrates an example display including a listing of alerts and a listing of text message conversations.

FIG. 32 illustrates an example display including a listing of alerts.

FIG. 33 illustrates an example display including a listing of alerts with a particular alert expanded.

FIG. 34 illustrates an example display including a box allowing the clinician to acknowledge an alert, forward an alert, or view a profile of the perinatal patient.

FIG. 35 illustrates an example display requesting confirmation of acknowledgement of an alert.

FIG. 36 illustrates an example display showing a listing of messages to the clinician.

FIG. 37 illustrates an example display displaying an option to forward notifications.

FIG. 38 illustrates an example display where all of blood pressure data, heart rate data, depression score data, social health score data, and a text message conversation listing are all displayed.

FIG. 39 illustrates an example display displaying depression score history information.

FIG. 40 illustrates an example display displaying search results of a search for a perinatal patient.

FIG. 41 illustrates an example display displaying a listing of text message conversations.

FIG. 42 illustrates an example display including sliderbars allowing a clinician to enter ranges for depression score, social health score, and blood pressure for which a notification (e.g., an alert) will be sent to the clinician.

FIG. 43 shows an example method of monitoring a perinatal patient subsequent to a childbirth event.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The present embodiments relate to, inter alia, tech-enabled medical monitoring of perinatal patients, and recommending treatments and/or clinician appointments for perinatal patients.

Following childbirth, the patient may experience significant physical and physiological medical conditions. For example, postpartum depression, which often goes undetected or untreated, harms many mothers. Sadly, some such medical conditions are even worse among certain racial minority populations.

To solve these problems and others, some embodiments gather medical data of a perinatal patient, e.g., a pregnant patient, a postpartum patient that has recently given birth, etc. In some examples, the perinatal patient is a patient that is monitored, as described herein, from the diagnosis of pregnancy to twelve months following childbirth. Additionally or alternatively, answers to depression survey questions, answers to social health survey questions, and/or answers to quality of life survey questions may be gathered; and a depression score and/or social health score of the perinatal patient may be determined. Any or all of the medical data, depression score, social health score, quality of life survey score, etc., may be used to trigger alerts to a clinician or a community-based partner local to the patient when patient intervention is needed, and/or make a recommendation (e.g., a recommended treatment, a recommendation for an appointment with a clinician, etc.). For instance, medical data exceeding thresholds or ranges generally, or thresholds or ranges associated with the particular patient, may trigger an alert to a clinician. Furthermore, combinations of medical data and one or more survey question answers may trigger an alert to a clinician. Additionally, in some examples, certain answers to certain survey questions may trigger an alert to a community-based partner local to the patient.

In some examples, this medical data includes biometric data, which may include weight data captured via a smart scale that a patient may use within their home (i.e., outside of a clinical setting), as well as blood pressure data captured via a smart blood pressure monitoring cuff, such as, e.g., a Microlife BT BP Cuff, which is FDA cleared and indicated for use in pregnant, preeclamptic and postpartum women within the home, i.e., outside of a clinical setting.

Various additional or alternative sensors may be used in some embodiments. In some examples, these sensors may include a glucose sensor, a sensor associated with an implantable device for measuring pre-term labor, or any other sensor suitable for monitoring perinatal patients.

For instance, in one example, an optical sensor (i.e., a light sensor) may be used to measure blood pressure and/or heart rate. In addition, some embodiments achieve specific improvements for certain racial minority patients. In one such example, some embodiments, to measure blood pressure and/or heart rate, use a pressure sensor (e.g., a Micro-Electro-Mechanical Systems (MEMS) sensor, etc.), rather than an optical sensor. This may improve data quality of the measured blood pressure and/or heart rate data for patients with dark skin tones. In another example, one or more machine learning algorithms are trained specifically for racial minority populations.

It should be appreciated that one example of a social health survey is a social determinants of health (SDoH) survey.

### Example System

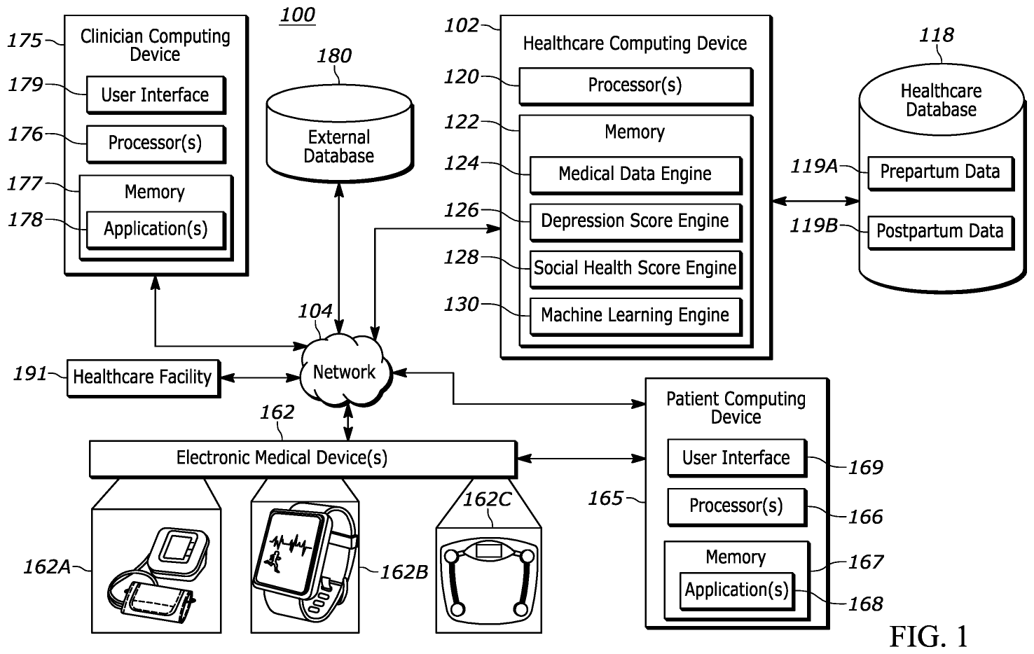

To this end, FIG. 1 illustrates an example perinatal monitoring and recommendation architecture 100, in accordance with an embodiment.

Broadly speaking, the architecture 100 may provide improved healthcare services and community-based partner services to postpartum patient (e.g., a patient who has recently given birth, a patient who has been diagnosed as pregnant, etc.). In some embodiments, the healthcare services are provided to the perinatal patient for a predetermined period of time following childbirth (e.g., for 6 months after childbirth, 1 year after child birth, 2 years after child birth, etc.).

The perinatal patient may use a perinatal patient computing device 165. The perinatal patient computing device 165 may be any suitable device. Examples of the perinatal patient computing device 165 include a smartphone, a tablet, a personal computer, a phablet, a smartwatch, etc. The patient computing device 165 may include one or more wired or wireless communication interfaces for communicating with any of the other devices shown in FIG. 1, one or more processors 166 such as one or more microprocessors, controllers, and/or any other suitable type of processor, and a memory 167 (e.g., volatile memory, non-volatile memory) accessible by the one or more processors 166, (e.g., via a memory controller). The memory 167 may store instructions that, when executed by the one or more processors, cause the one or more processors to execute one or more application(s) 168. The patient computing device 165 may further include a user interface 169, via which the patient computing device 165 may receive inputs from the patient and/or present information to the patient (e.g., audibly via one or more speakers, visually via a screen display, etc.).

The perinatal patient may also use one or more electronic medical device(s) 162. The electronic medical device(s) 162 may include any suitable device or sensor worn or otherwise used for capturing health data associated with the patient. Examples of the electronic medical device 162 include a pressure sensor (e.g., to measure blood pressure and/or heart rate, such as a Micro-Electro-Mechanical Systems (MEMS) sensor), a blood pressure monitoring cuff 162A (such as, e.g., a Microlife BT BP Cuff, which is FDA cleared and indicated for use in pregnant, preeclamptic and postpartum women within the home, i.e., outside of a clinical setting), a smartwatch 162B (e.g., a smartwatch that gathers blood pressure, and/or heart rate data), a smart scale 162C, a glucose monitoring device, an implantable sensor for capturing data related to pre-term labor, etc. The electronic medical device(s) 162 may include fixed or mobile devices in various embodiments, and may include devices that may be used outside of clinical settings in a patient's home. In some examples, advantageously, the electronic medical device 162 is a wearable device. This beneficially allows the electronic medical device 162 to collect any data continually or periodically (e.g., even when the perinatal patient is not in a clinical setting, such as a hospital).

The electronic medical device 162 may include one or more wired or wireless communication interfaces for communicating with any of the other devices shown in FIG. 1. For example, in some embodiments, the electronic medical device 162 is directly communicatively coupled with the perinatal patient computing device 165 (e.g., via Bluetooth, etc.). Additionally or alternatively, the electronic medical device 162 and the perinatal patient computing device 165 may communicate via the network 104, which may be any suitable network, such as the internet. It should further be appreciated that the network 104 may be any suitable wireless or wired network, and that any of the components of the example architecture 100 may communicate via the network 104.

In some embodiments, the electronic medical device 162 and/or perinatal patient computing device 165 may send data to the healthcare computing device 102. Examples of the data sent to the healthcare computing device 102 include data of the perinatal patient, and answers to survey questions (e.g., depression survey questions, social health score survey questions, quality of life survey questions such as SF-36 survey questions, etc.). Furthermore, an application 168 of the patient computing device 165 may send messages input by the patient (e.g., via a user interface 169 of the patient computing device 165) to the clinician computing device 175, discussed in greater detail below, and the application 168 of the patient computing device 165 may in turn receive messages from the clinician computing device 175, e.g., messages input by the clinician via a user interface of the clinician computing device 175.

In this way, the patient or a caregiver of the patient may communicate with the clinician in order to ask questions or provide updates related to the patient's care. For instance, the patient may send a message, via the application 168 of the patient computing device 165, indicating that she is experiencing a particular condition, and the clinician may receive the message, via the clinician computing device 175, and respond with an indication of whether/how the patient should address the condition from home, come into the office, schedule an appointment, etc. The application 168 of the patient computing device 165, and/or an application 178 of the clinician computing device 175, may store the messages exchanged between the patient computing device 165 and the clinician computing device 175 (e.g., on the memory 167, on the memory 177, or on an external database), as well as indications of times associated with each message.

In some examples, the patient computing device 165 and/or the clinician computing device 175 may correlate the times associated with messages sent by the patient with corresponding measurements captured by the electronic medical device 162 at the same time (or at proximate times, e.g., within a threshold period of time from the time the message is received or sent). For instance, these time-correlated messages and measurements may be analyzed in order to identify patient-reported conditions associated with certain measurements captured by the electronic medical device 162. Additionally, in some examples, the patient may send a message to the clinician computing device 175, via the application 168 of the patient computing device 165, including image data associated with a condition the patient is experiencing. For instance, the image may include an image of a patient wound (such as a C-section incision), a patient skin condition, or other visual data associated with a condition the patient is experiencing.

In some examples the patient computing device 165 and/or the clinician computing device 175 (or another backend computing device, such as the healthcare computing device 102) may analyze the image data (in some cases, in conjunction with the text of the message, and/or any data captured by the electronic medical device 162 at proximate times) in order to identify a patient condition. For instance, a particular word or phrase in the message, in conjunction with an image with one or more visually discernible features, and/or one or more measurements captured by the electronic medical device 162 (such as, e.g., a spike in heart rate, blood pressure, etc.), may be correlated with a particular condition, disease, state, etc., of the patient.

In some examples, the patient computing device 165 and/or the clinician computing device 175 (or another backend computing device, such as the healthcare computing device 102) may determine a severity associated with the message sent by the patient based on the text of the message, the image included in the message, and/or any data captured by the electronic medical device 162 at proximate times to the times at which the messages are sent and/or times at which images are captured (which may be determined, e.g., based on metadata associated with the images). When providing the patients' messages to the clinician, an application 178 of the clinician computing device 175 that provides messages from the patient to the clinician may prioritize any messages identified as being greater than a threshold level of severity. For instance, the application 178 may cause the user interface 179 of the clinician computing device 175 to visually highlight or flag, or generate audible alerts for, any messages from the patient that are identified as being associated with patient conditions having a severity level that is greater than the threshold severity level. Moreover, in some examples, any messages identified as having greater than a higher threshold level of severity may be routed immediately to emergency services (e.g., an emergency room, an ambulance, a 911 number, etc.), or may be routed to emergency services if the clinician does not respond within a threshold period of time.

Furthermore, in some examples, an application 168 of the patient computing device 165 and/or an application 178 of the clinician computing device 175 may track the time it takes the clinician to respond to patient messages. This tracked time may be stored on both a per-clinician and per-patient basis, for various levels of message severity, in order to identify any clinicians who lag in responding to patients generally, any patients who clinicians lag in responding to compared to other patients, and/or any combination of the two (i.e., particular patients that particular clinicians lag in responding to), as well as any clinicians who lag in responding to higher severity messages in any of these categories. This data may be tracked and stored on the memories 167 or 177, or on a memory 122 of a backend healthcare computing device 102, or in an external database. One or more of the application 168 or application 178, or an application stored on the memory 122 of the backend healthcare computing device 102 may trigger a notification, an intervention, an investigation, or other mitigating actions for clinicians identified as lagging in responding to patients generally, any patients who clinicians lag in responding to compared to other patients, and/or any combination of the two (i.e., particular patients that particular clinicians lag in responding to), as well as any clinicians who lag in responding to higher severity messages in any of these categories. In this manner, patient health outcomes may be improved, as clinicians who lag in responsiveness are identified and corrected.

Generally speaking, data of the perinatal patient may be any suitable data for monitoring the health and well-being of the perinatal patient. Examples of the data of the perinatal patient include blood pressure data (e.g., systolic blood pressure data, and/or diastolic blood pressure data) of the perinatal patient, heart rate data of the perinatal patient, weight data of the perinatal patient, height data of the perinatal patient, racial data of the perinatal patient, age data of the perinatal patient, socioeconomic data of the perinatal patient, address data of the perinatal patient, medical history data of the perinatal patient, blood oxygen data of the perinatal patient, and/or respiration data of the perinatal patient. Moreover, any of the data may include timestamps (e.g., timestamps indicating when a medical measurement was taken, and/or when the data was captured by and/or sent from the electronic medical device 162, etc.).

The healthcare computing device 102 may receive the data, and may use the data in accordance with the techniques discussed herein (e.g., to provide recommendations to the perinatal patient and/or clinician, etc.). The healthcare computing device 102 may include one or more wired or wireless communication interfaces for communicating with any of the other devices shown in FIG. 1, one or more processors 120 such as one or more microprocessors, controllers, and/or any other suitable type of processor, and a memory 122 (e.g., volatile memory, non-volatile memory) accessible by the one or more processors 120, (e.g., via a memory controller).

The one or more processors 120 may interact with the memory 122 to obtain, for example, computer-readable instructions stored in the memory 122. Additionally or alternatively, computer-readable instructions may be stored on one or more removable media (e.g., a compact disc, a digital versatile disc, removable flash memory, etc.) that may be coupled to the healthcare computing device 102 to provide access to the computer-readable instructions stored thereon. In particular, the computer-readable instructions stored on the memory 122 may include instructions for executing various applications, such as the medical data engine 124, depression score engine 126, social health score engine 128, and/or machine learning engine 130.

In operation, the healthcare computing device 102 enhances a clinician's ability to provide healthcare services to the perinatal patient. The clinician may be any kind of clinician. Examples of clinicians may include a primary care provider (i.e., a physician), a social worker, a nurse, etc. Furthermore, the clinician may have a clinician computing device 175, such as a smart phone, a personal computer, a tablet, a phablet, etc. Although one clinician computing device 175 is shown in FIG. 1, in some examples, multiple such clinician computing devices 175 may be included, such that multiple healthcare providers are equipped to monitor data associated with the patient (e.g., for redundancy if one healthcare provider is not responsive to the patient).

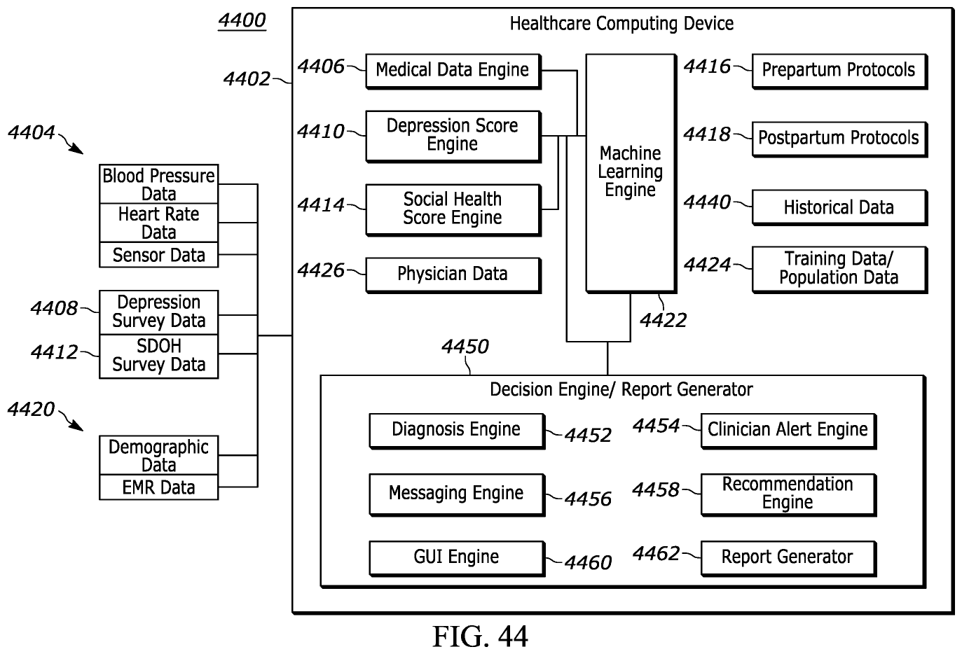

The clinician computing device 175 may include one or more processors 176 such as one or more microprocessors, controllers, and/or any other suitable type of processor. The clinician computing device 175 may further include a memory 177 (e.g., volatile memory, non-volatile memory) accessible by the one or more processors 176, (e.g., via a memory controller). The memory 177 may store instructions that, when executed by the one or more processors, cause the one or more processors 176 to perform one or more of the steps discussed with respect to the method 4300 at FIG. 43. Moreover, the memory 177 may store instructions that, when executed by the one or more processors 176, cause the one or more processors 176 to execute one or more application(s) 178. The clinician computing device 175 may further include a user interface 179, via which the clinician computing device 175 may receive inputs from the clinician and/or present information to the clinician (e.g., audibly via one or more speakers, visually via a screen display, etc.), including messages, images, measurements, etc., as discussed above with respect to the application 168. Furthermore, in some examples, the clinician computing device 175 may additionally include a wired or wireless communication interface to facilitate communication with the other devices shown at FIG. 1, such as, e.g., the healthcare computing device, e.g., via the network 104.

In this regard, as will be further discussed below, the healthcare computing device 102 may present information of the perinatal patient, and or recommendation(s) to the clinician. For example, any of the data of the perinatal patient may be presented to the clinician. Additionally or alternatively, a depression score (e.g., determined by the depression score engine 126), and/or a social health score (e.g., determined by the social health score engine 128) may be presented to the clinician (e.g., via the clinician computing device 175, etc.). Additionally or alternatively, the clinician may be presented with a recommendation for the perinatal patient (e.g., a recommendation for a treatment, a recommendation for a clinician appointment, etc.). Furthermore, the computing device 102 may present the clinician with an alert associated with the perinatal patient. The alert may indicate that intervention is required, and may be triggered based on the patient's medical data and/or answers to various questions, individually or in combination, exceeding one or more general ranges or thresholds, or one or more ranges or thresholds particular to the patient. It should be appreciated that any of the presentations made to the clinician may be made via the clinician computing device 175.

Moreover, although the computing device 102 is discussed herein as a “healthcare computing device” and the device 175 is discussed herein as a “clinician computing device 175,” in some embodiments, the computing device 102 and/or the device 175 may be devices associated with community-based partners who are not necessarily healthcare oriented, and/or who are not necessarily clinicians (e.g., local charities, government organizations, social programs, etc.), in a similar manner. For instance, based on patient's answers to various survey questions (such as answers indicative of depression, answers indicative of low social determinants of health, answers indicative of low quality of life, etc.), a computing device 102 and/or a device 175 associated with a local community-based partner may trigger an alert indicating that patient intervention is needed. The community-based partner may then reach out to the patient (e.g., via the patient computing device 165) to alleviate issues indicated by the patient's answers to the survey questions. For example, while confidential patient medical data may be communicated only with the appropriate clinician, other patient issues may be addressed by community-based partners, such as if a patient needs assistance from a social worker, assistance with obtaining a ride to a medical appointment, assistance with affording diapers, etc.

Additionally or alternatively, the healthcare computing device 102 may receive data (e.g., data of the perinatal patient, etc.) from other sources, such as the external database 180 (e.g., a medical records database, etc.), healthcare facility 191, etc. The healthcare facility 191 may be any kind of healthcare facility 191, such as a hospital, an urgent care facility, a clinic, a birthing center, etc.

Furthermore, the healthcare computing device 102 may store any data in the healthcare database 118, including the obtained prenatal data 119A and/or postpartum data 119B. Examples of the prenatal data 119A and/or postpartum data 119B include healthcare data (blood pressure, heart rate data, etc.), depression data, social health data, and any other data measured by or otherwise provided to the system.

In some examples, the prenatal data 119A and/or the postpartum data 119B are part of historical data (e.g., data used to train a machine learning algorithm as discussed herein). Additionally or alternatively, the healthcare database 118 may store historical data of other patients besides the perinatal patient (e.g., historical data used to train a machine learning algorithm as discussed herein). Any of the data (e.g., the prenatal data 119A, the postpartum data 119B, the historical data, etc.) may additionally or alternatively be stored in the external database 180.

In addition, although the example system 100 illustrates only one of each of the components, any number of the example components are contemplated (e.g., any number of perinatal patients, clinicians, hospitals, healthcare computing devices, etc.).

### Example Displays—Perinatal Patient Computing Device

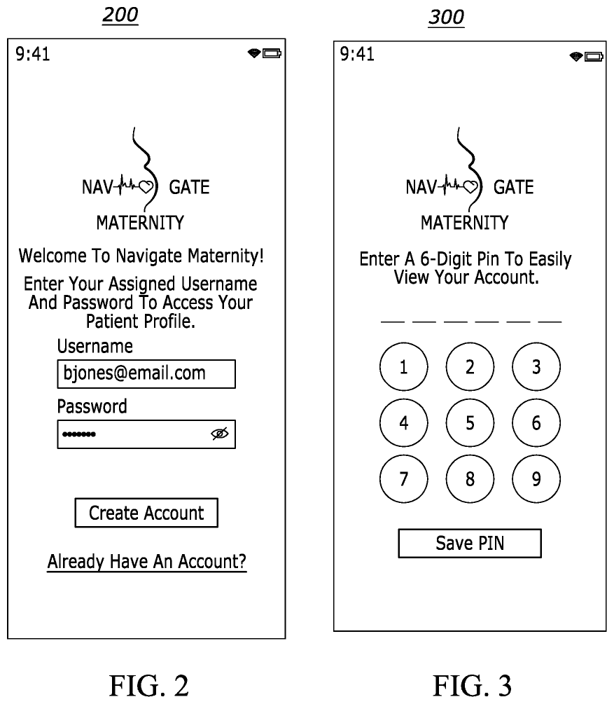

FIGS. 2-29 show examples of displays that may be presented to the perinatal patient (e.g., via the perinatal patient computing device 165 or any other suitable device).

FIG. 2 illustrates an example display 200 allowing the perinatal patient to login.

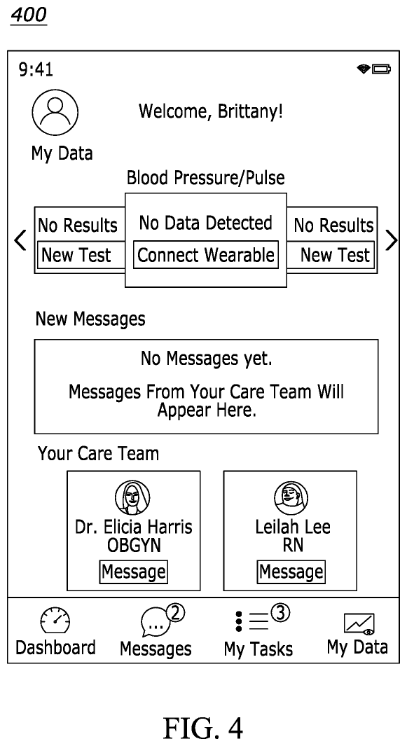

FIG. 3 illustrates an example display 300 allowing the perinatal patient to enter a personal identification number (PIN) as part of logging in.

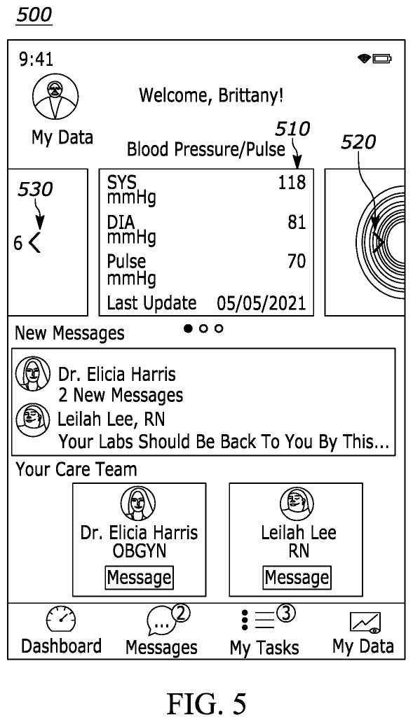

FIG. 4 illustrates an example display 400 where no patient data has been detected.

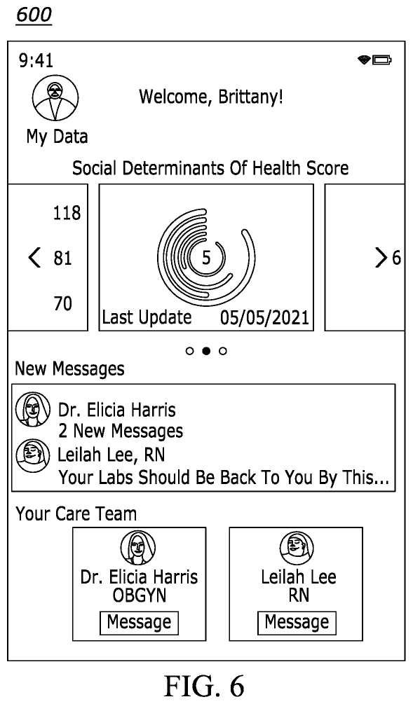

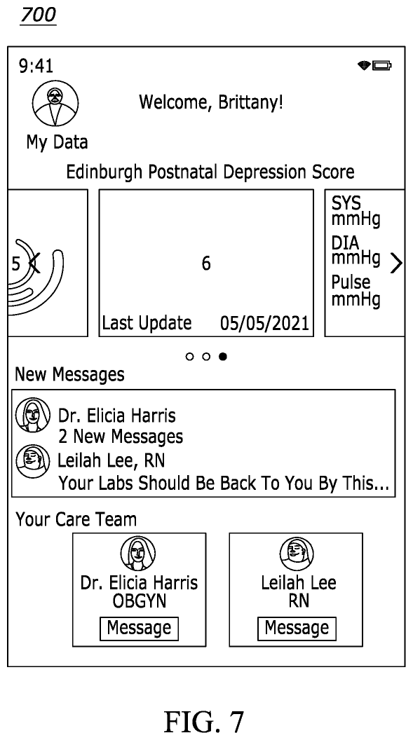

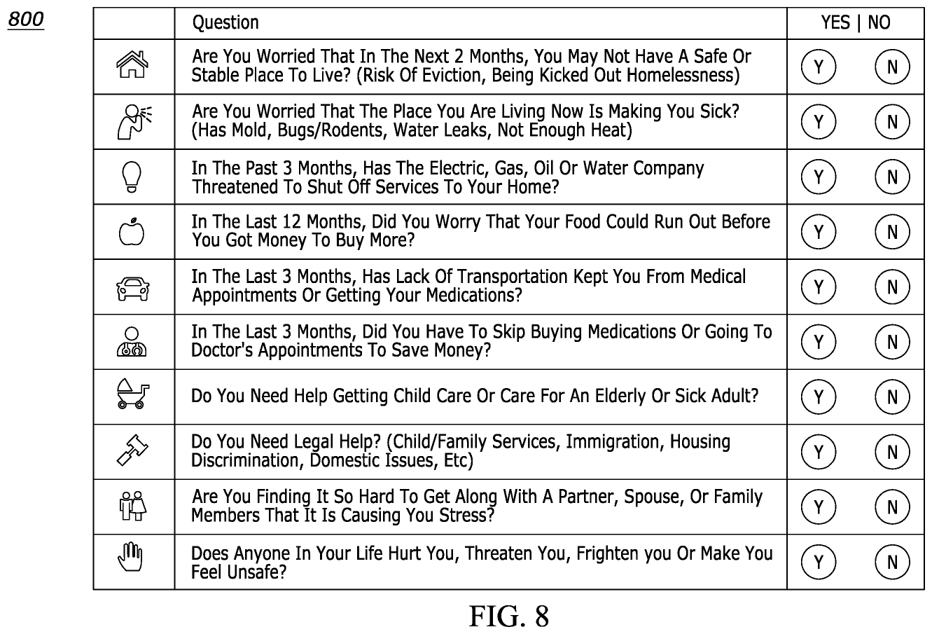

FIG. 5 illustrates an example display 500 populated with the perinatal patient's data, such as blood pressure data and heart rate data 510. The example display 500 also includes scroll button 520, which allows the perinatal patient to scroll to view the social health score (e.g., scroll to view the example display 600 of the example of FIG. 6). The example display 500 further includes scroll button 530, which allows the perinatal patient to scroll to view the depression score (e.g., scroll to view the example display 700 of the example of FIG. 7).

FIG. 6 illustrates an example display 600 displaying an example social health score.

FIG. 7 illustrates an example display 700 displaying an example depression score.

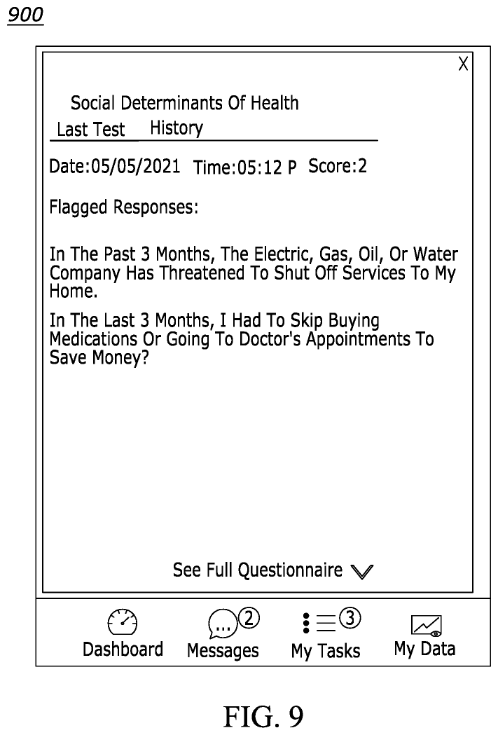

FIG. 8 illustrates an example display 800 displaying example social health survey questions.

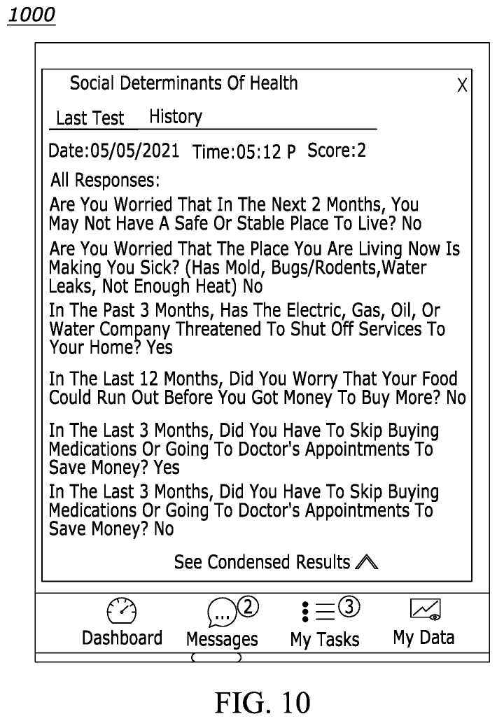

FIG. 9 illustrates an example display 900 displaying aspects of flagged responses of an example social health survey.

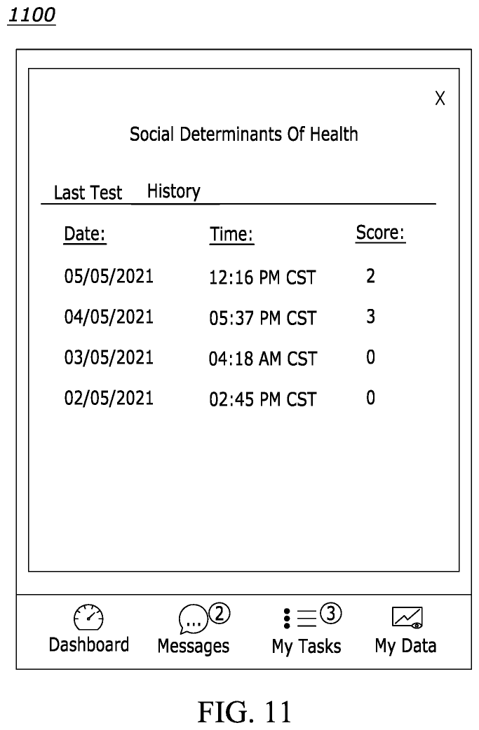

FIG. 10 illustrates an example display 1000 displaying example responses of an example social health survey.

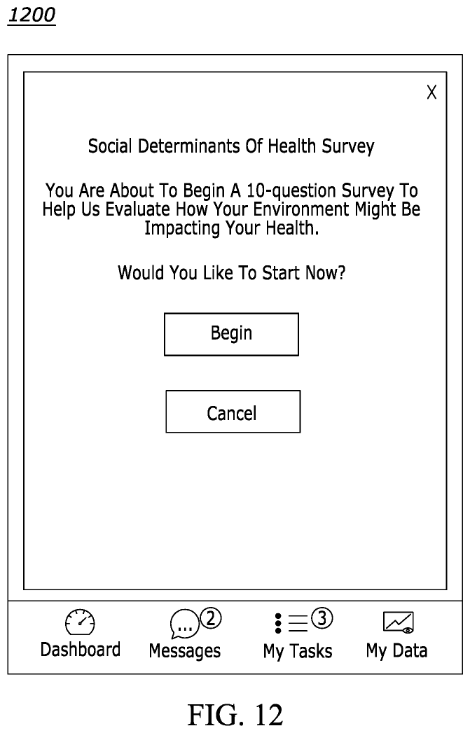

FIG. 11 illustrates an example display 1100 displaying example past scores of social health surveys.

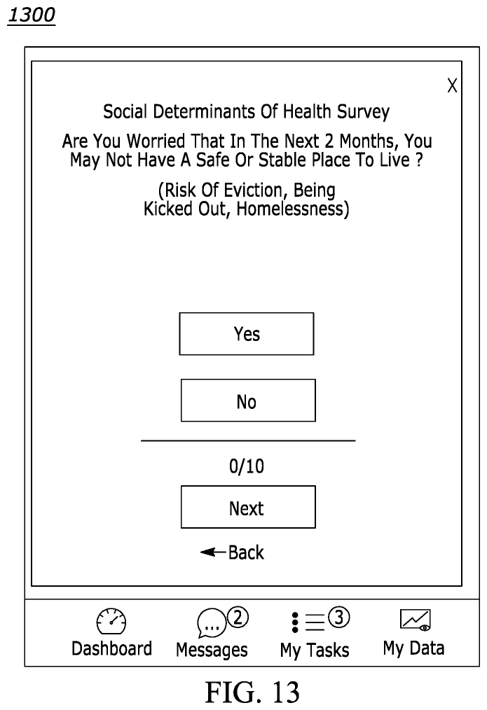

FIG. 12 illustrates an example display 1200 displaying an example beginning to an example social health survey.

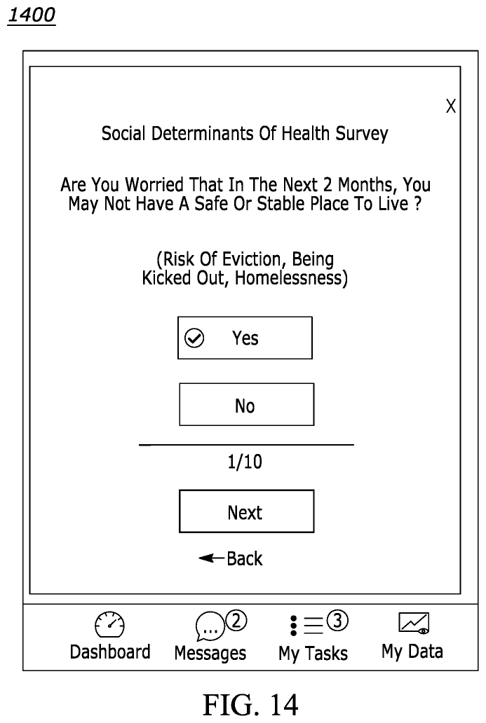

FIG. 13 illustrates an example display 1300 displaying an example social health survey question.

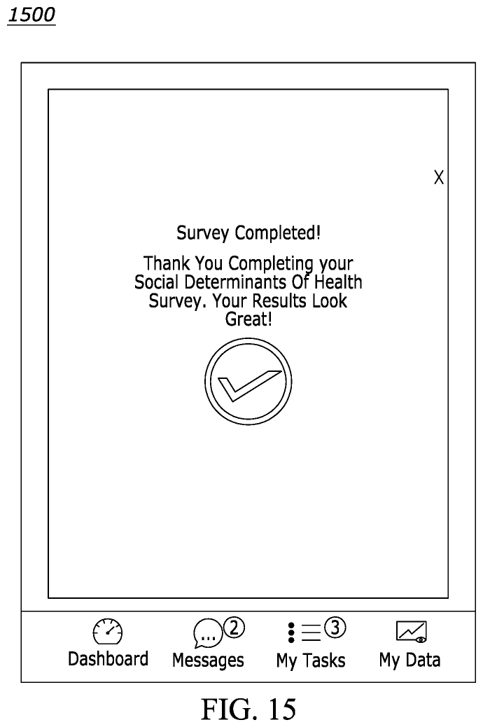

FIG. 14 illustrates an example display 1400 displaying an example social health survey question with an answer filled in.

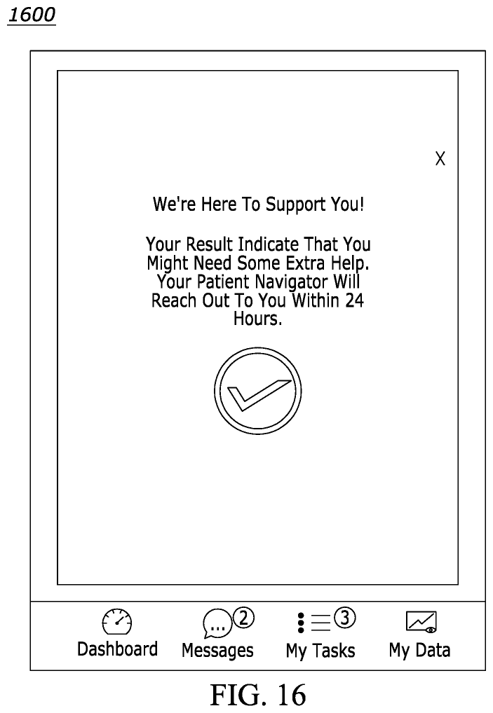

FIG. 15 illustrates an example display 1500 indicating that a social health survey is complete.

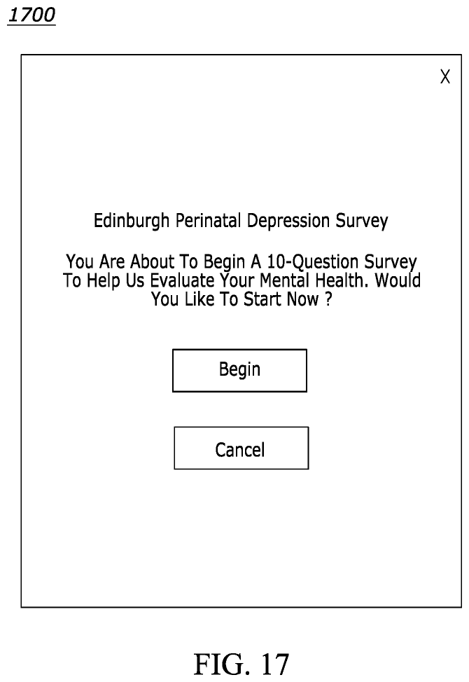

FIG. 16 illustrates an example display 0 including an alert that the perinatal patient may need additional help following a social health survey.

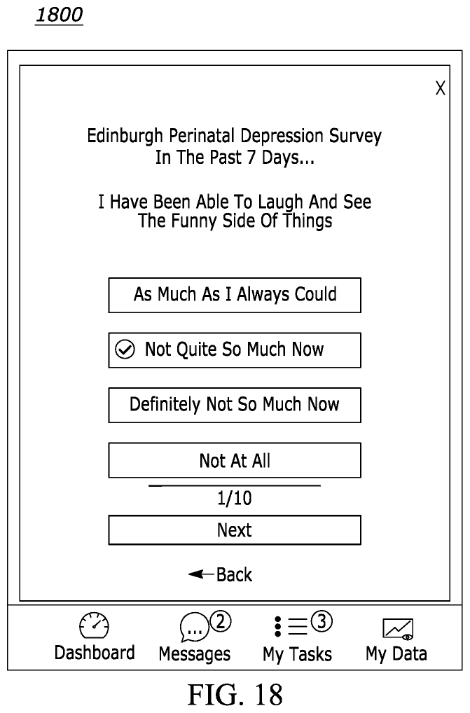

FIG. 17 illustrates an example display 0 displaying a beginning to an example depression survey.

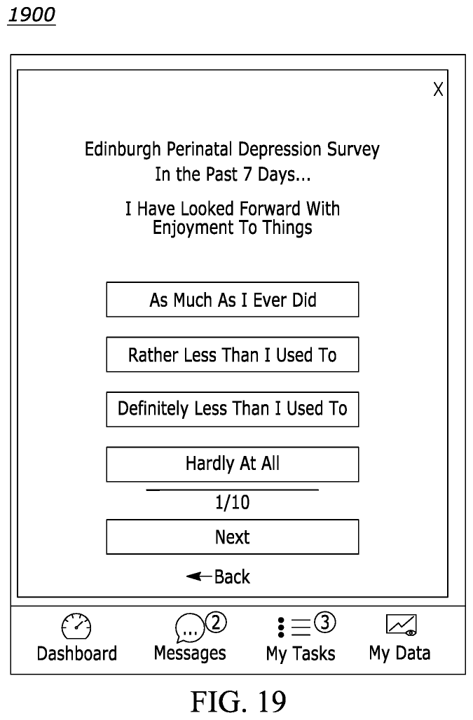

FIG. 18 illustrates an example display 1800 displaying an example depression survey question with an answer filled in.

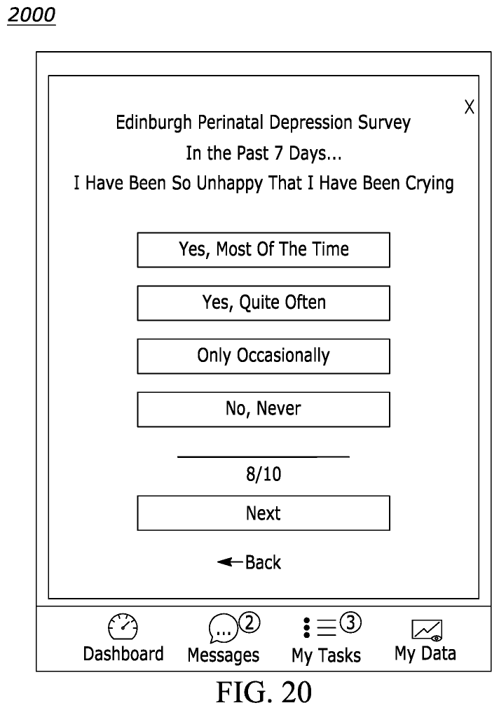

FIG. 19 illustrates an example display 1900 displaying an example depression survey question without an answer filled in.

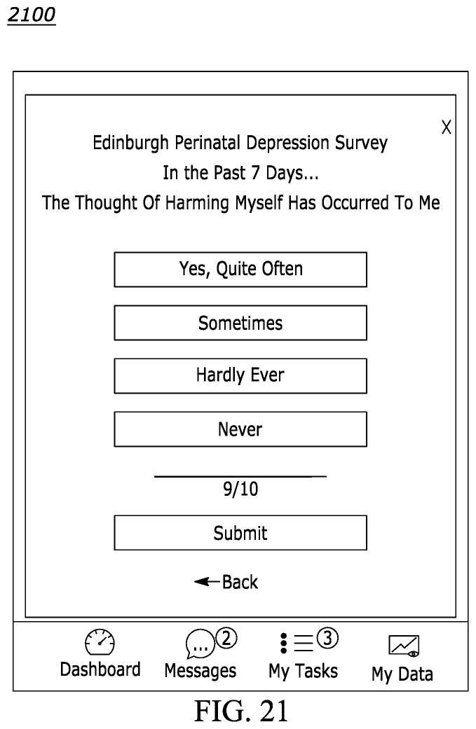

FIG. 20 illustrates an example display 2000 displaying another example depression survey question without an answer filled in.

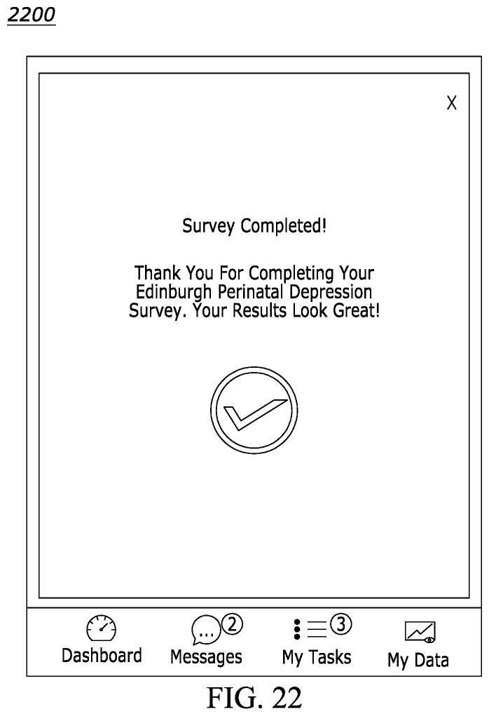

FIG. 21 illustrates an example display 2100 displaying another example depression survey question without an answer filled in.

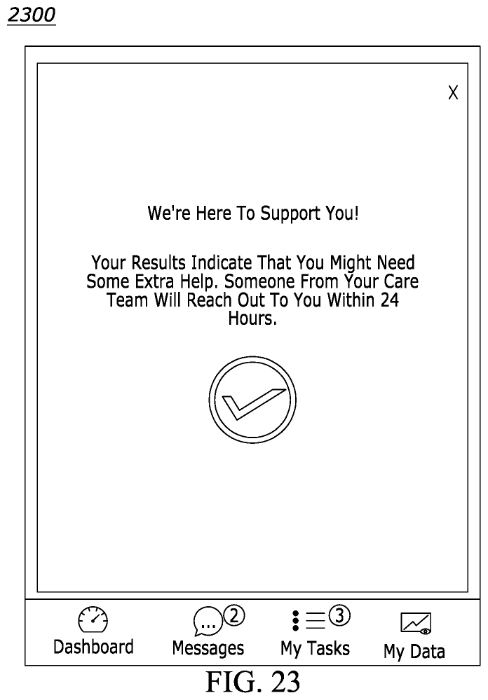

FIG. 22 illustrates an example display 2200 indicating that a depression survey is complete.

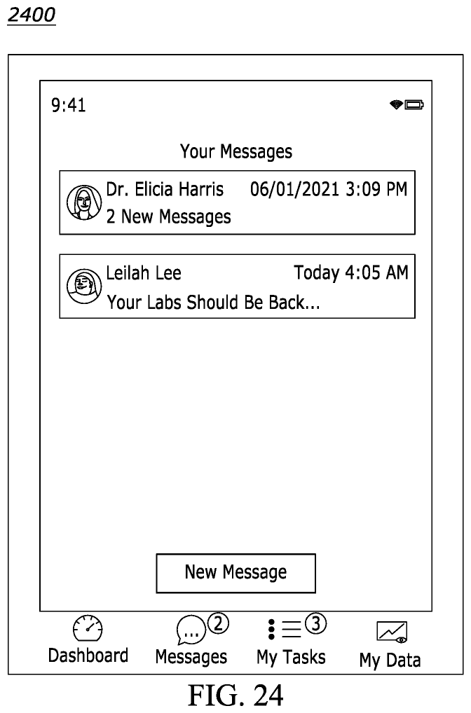

FIG. 23 illustrates an example display 2300 including an alert that the perinatal patient may need additional help following a depression survey.

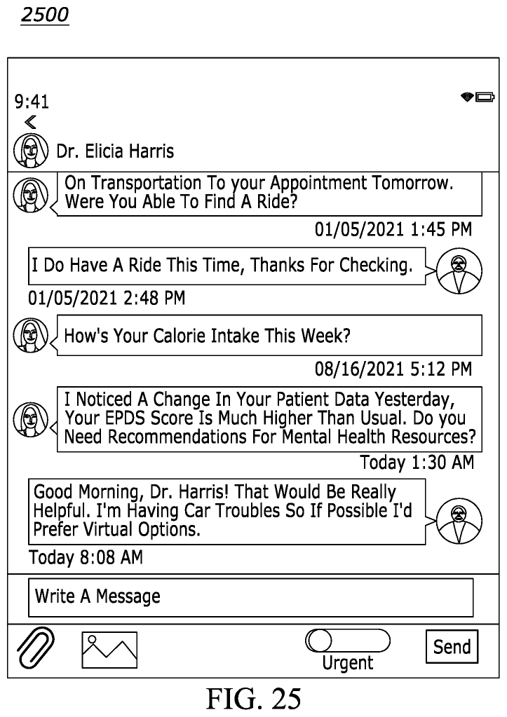

FIG. 24 illustrates an example display 2400 displaying a selection of text message conversations.

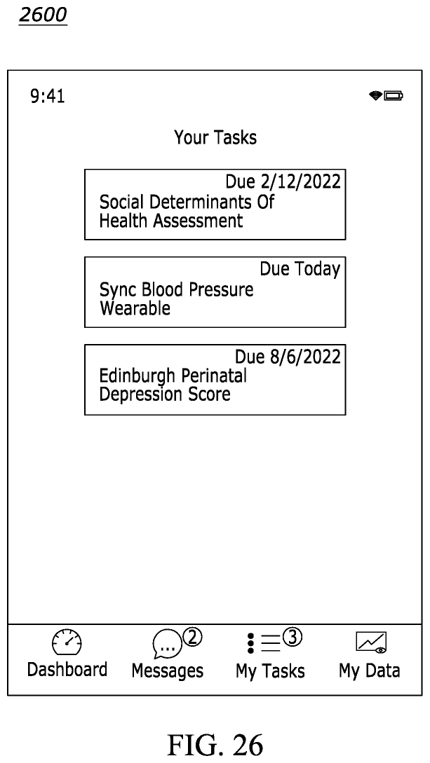

FIG. 25 illustrates an example display 2500 displaying a particular text message conversation.

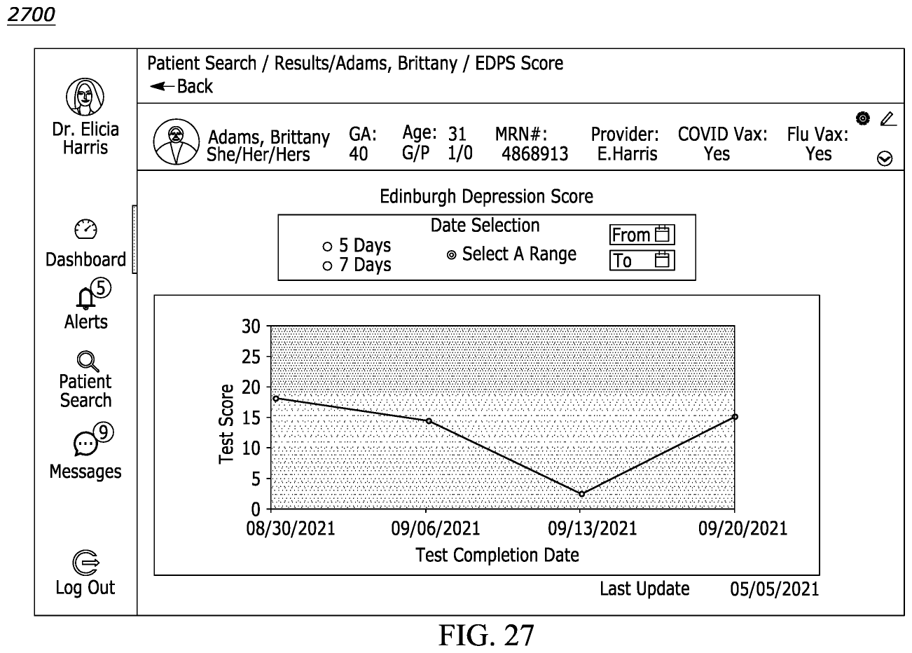

FIG. 26 illustrates an example display 2600 displaying a list of tasks.

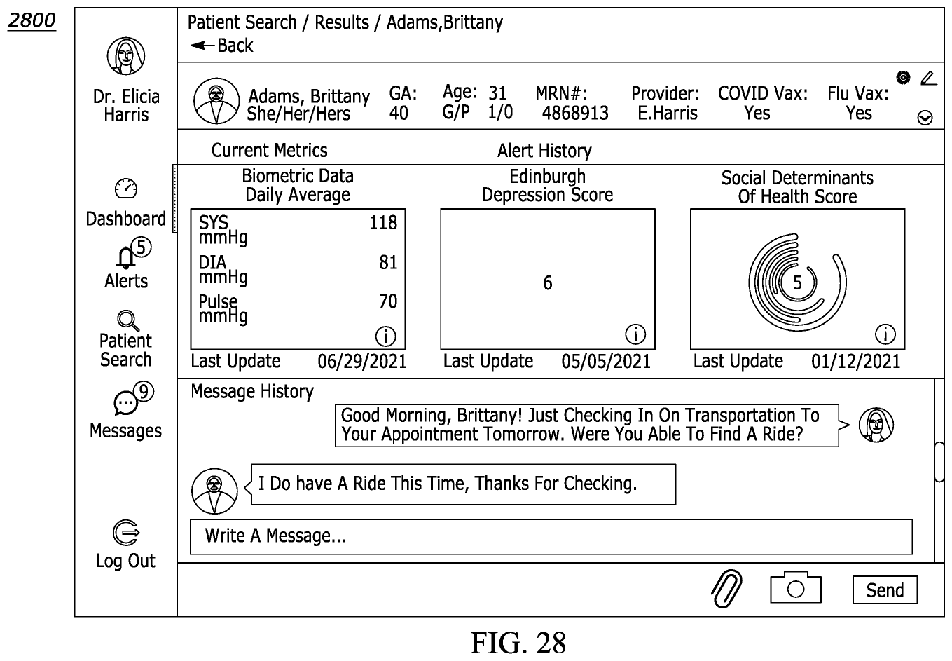

FIG. 27 illustrates an example display 2700 displaying depression survey and depression score information.

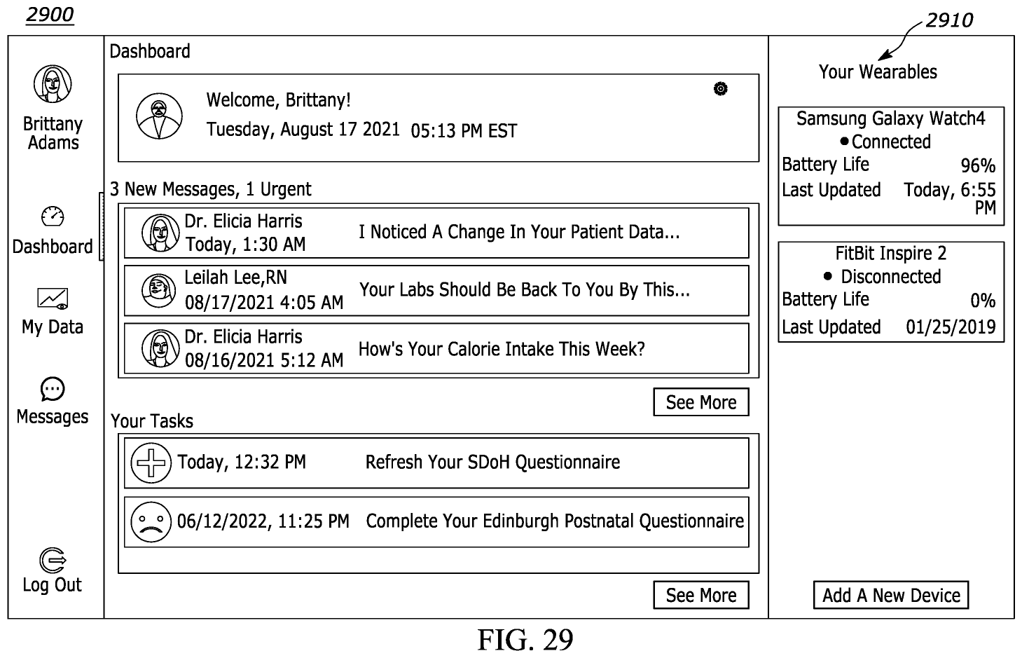

FIG. 28 illustrates an example display 2800 where all of blood pressure data, heart rate data, depression score data, social health score data, and a text message conversation are all displayed.

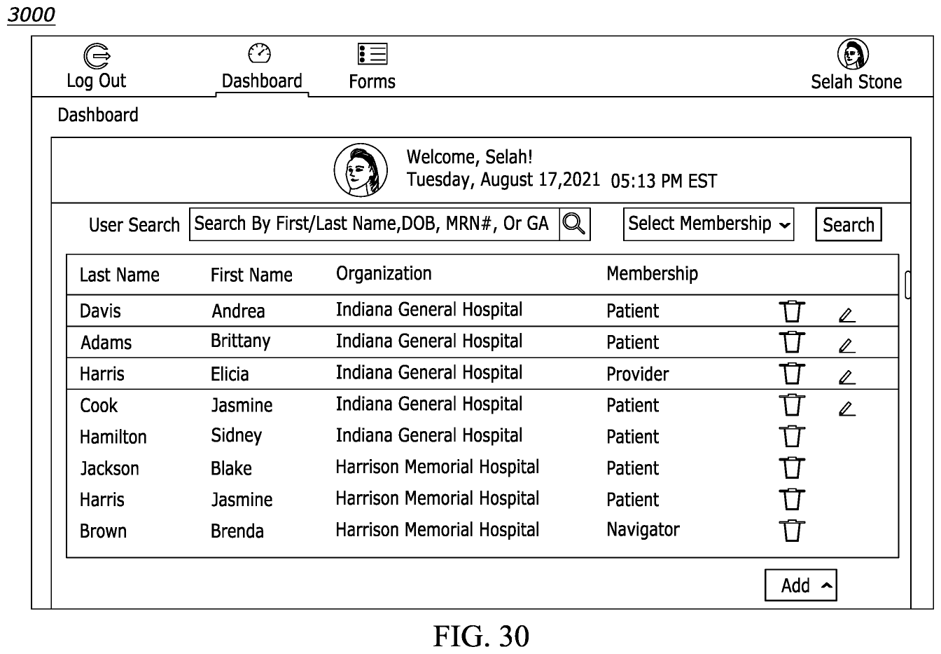

FIG. 29 illustrates an example display 2900 including a list 2910 of electronic medical device(s) 162.

### Example Displays—Clinician Computing Device

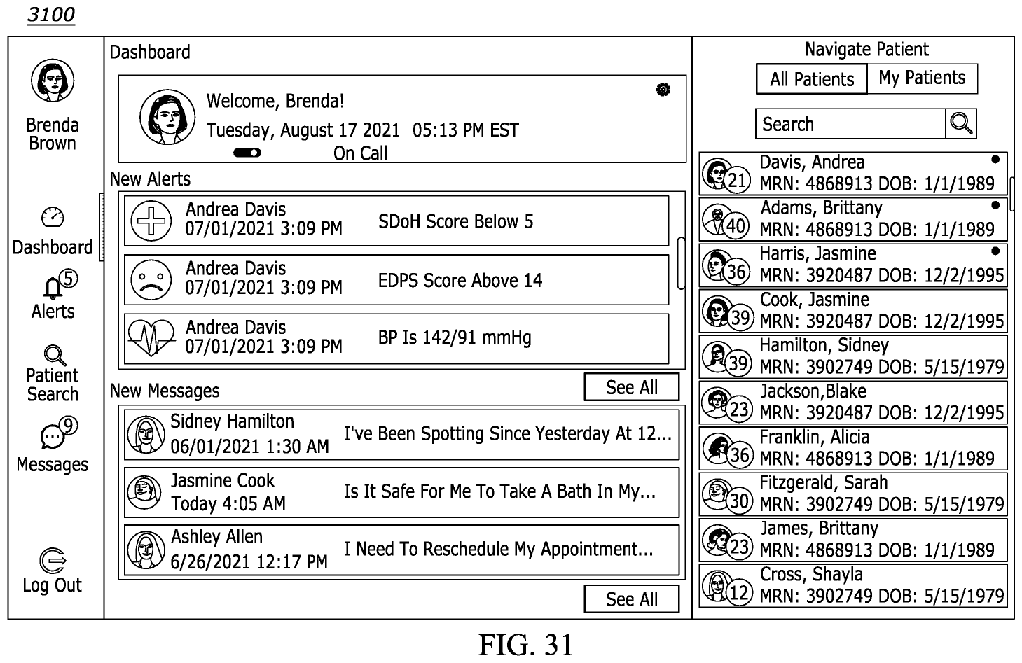

FIGS. 30-42 show examples of displays that may be presented to the clinician (e.g., via the clinician computing device 175 or any other suitable device).

FIG. 30 illustrates an example display 3000 allowing a clinician to search for a perinatal patient.

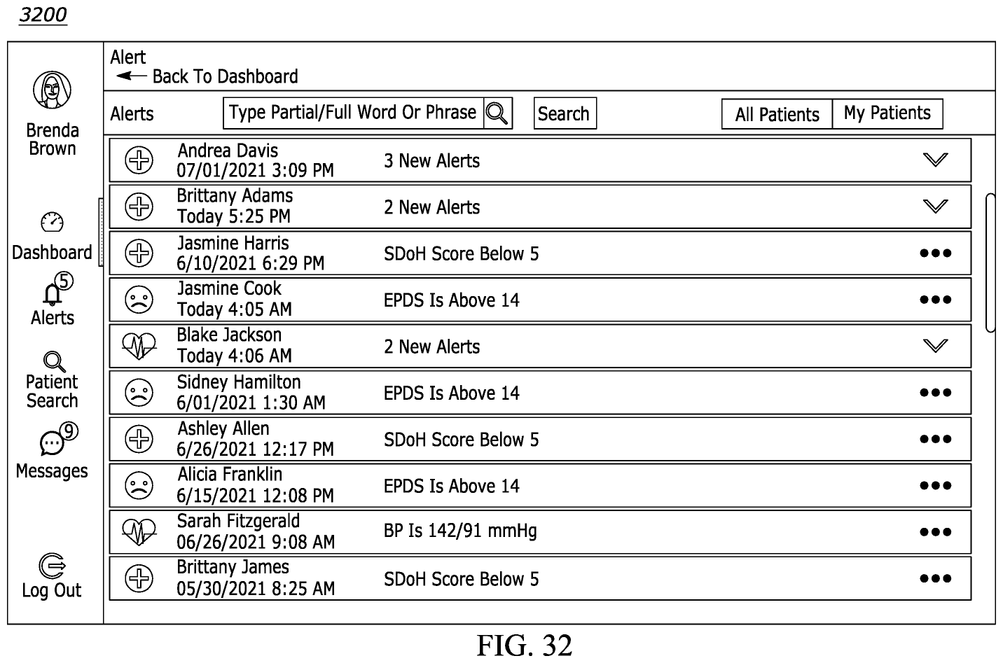

FIG. 31 illustrates an example display 3100 including a listing of alerts and a listing of text message conversations.

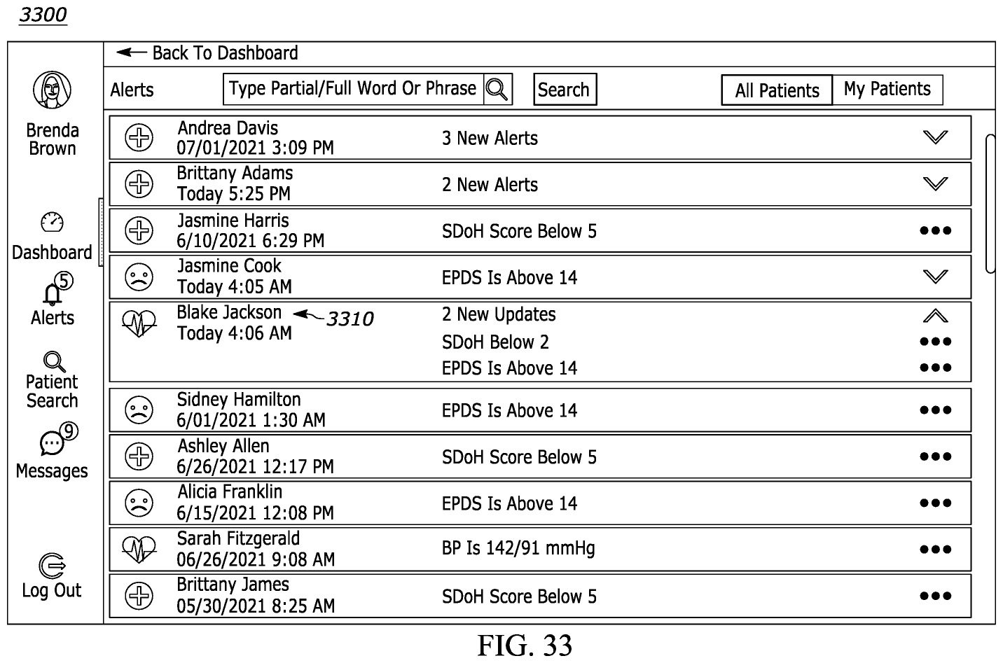

FIG. 32 illustrates an example display 3200 including a listing of alerts.

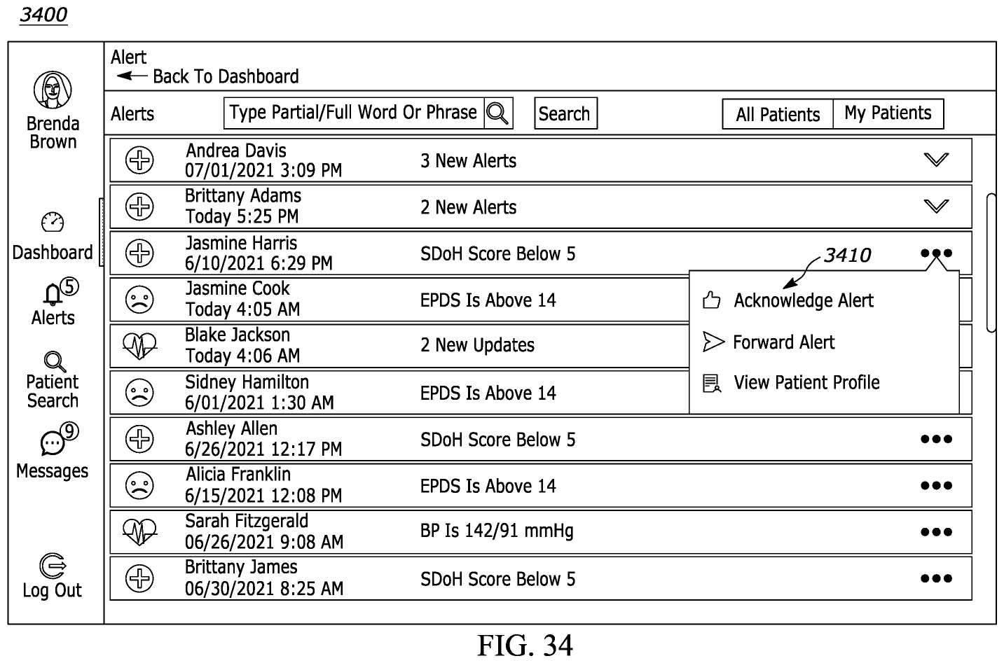

FIG. 33 illustrates an example display 3300 including a listing of alerts with a particular alert 3310 expanded.

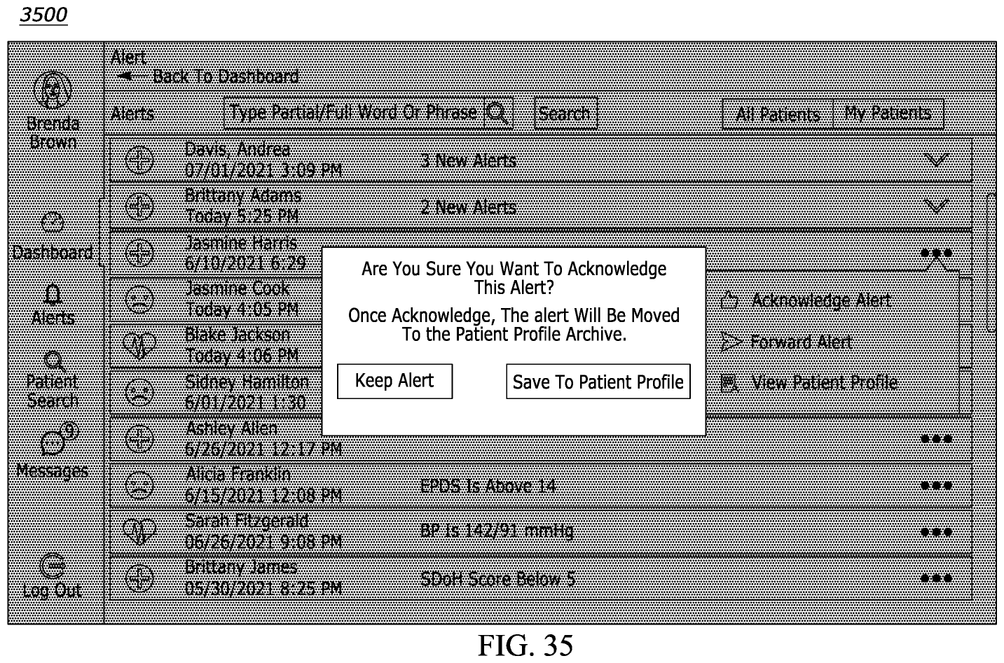

FIG. 34 illustrates an example display 3400 including a box 3410 allowing the clinician to acknowledge an alert, forward an alert, or view a profile of the perinatal patient.

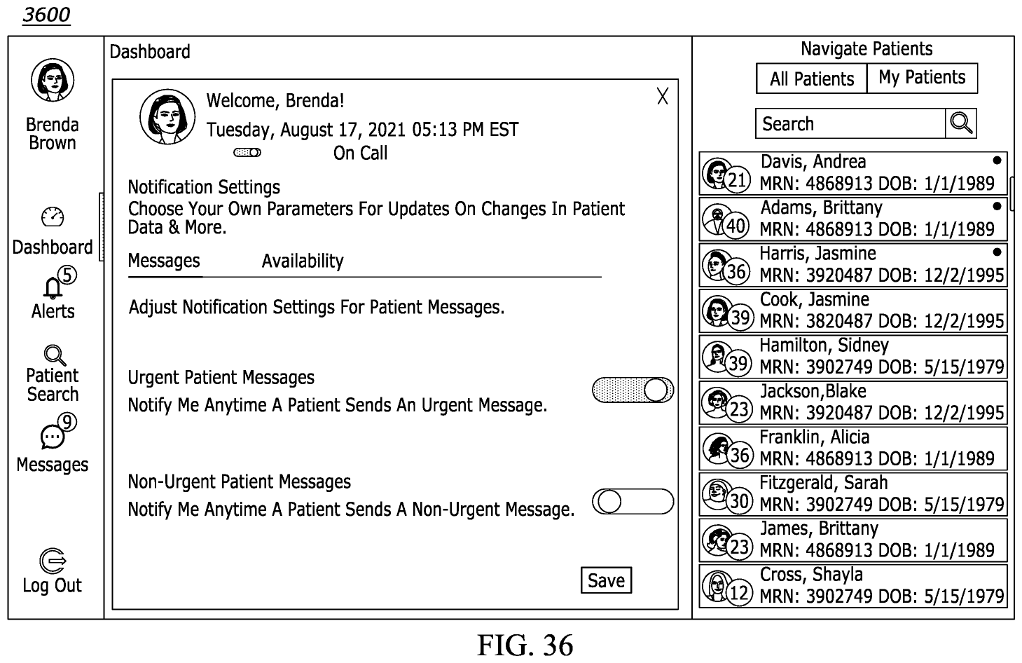

FIG. 35 illustrates an example display 3500 requesting confirmation of acknowledgement of an alert.

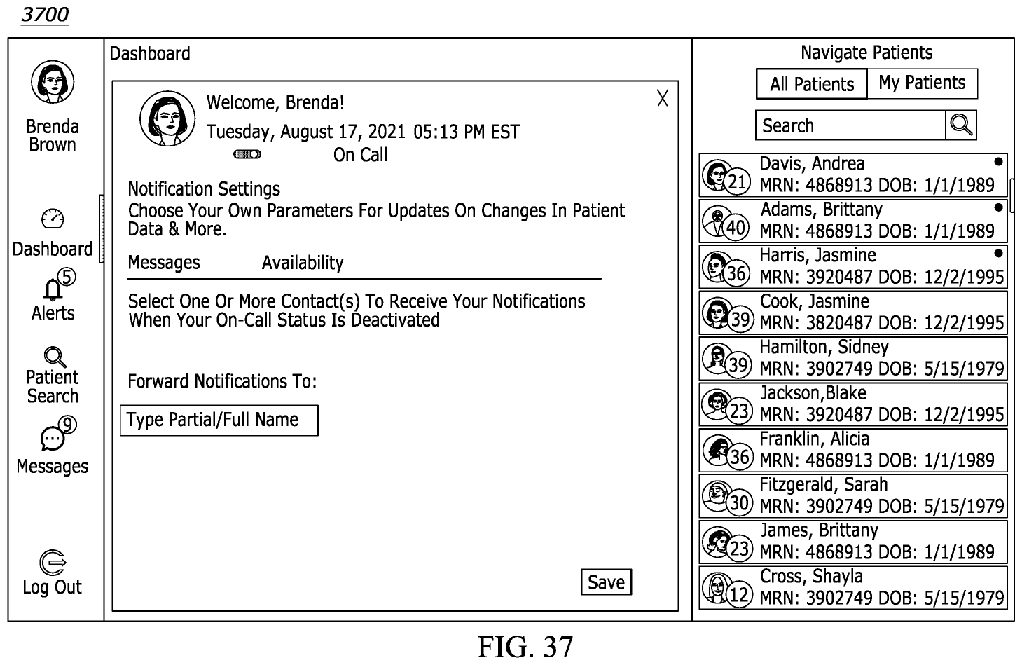

FIG. 36 illustrates an example display 3600 showing a listing of messages to the clinician.

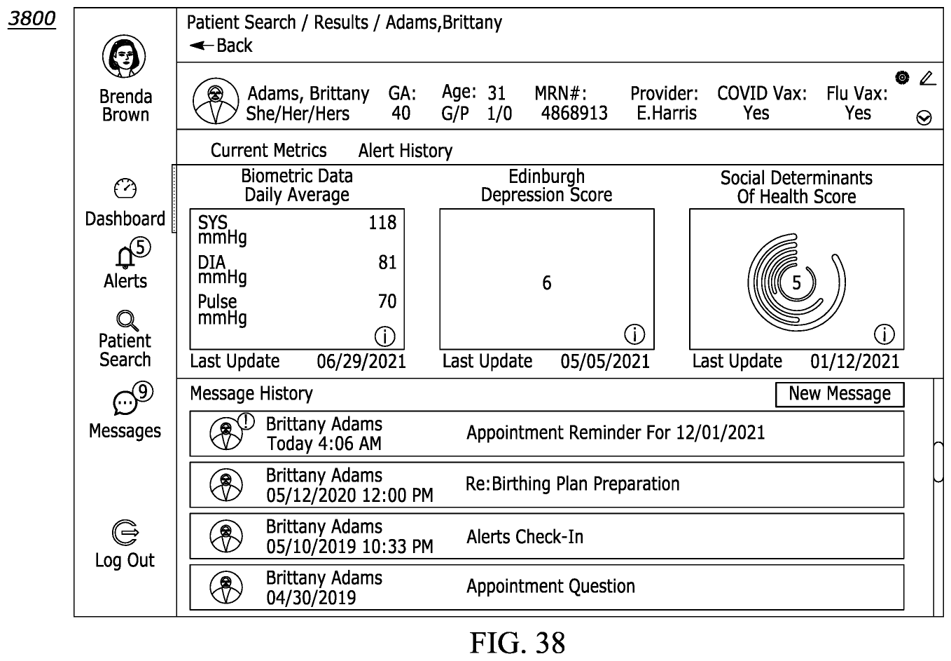

FIG. 37 illustrates an example display 3700 displaying an option to forward notifications.

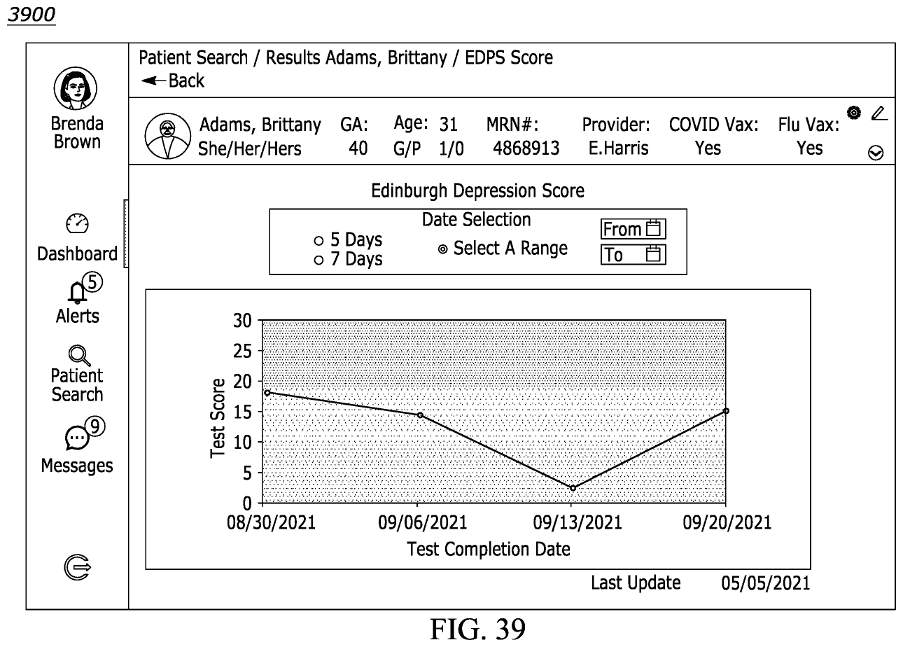

FIG. 38 illustrates an example display 3800 where all of blood pressure data, heart rate data, depression score data, social health score data, and a text message conversation listing are all displayed.

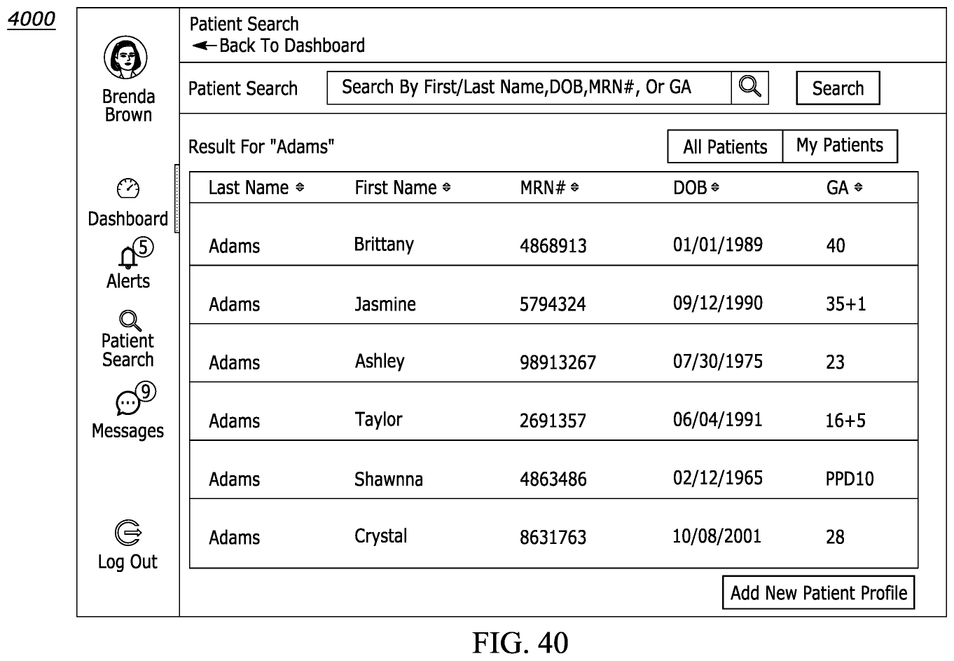

FIG. 39 illustrates an example display 3900 displaying depression score history information.

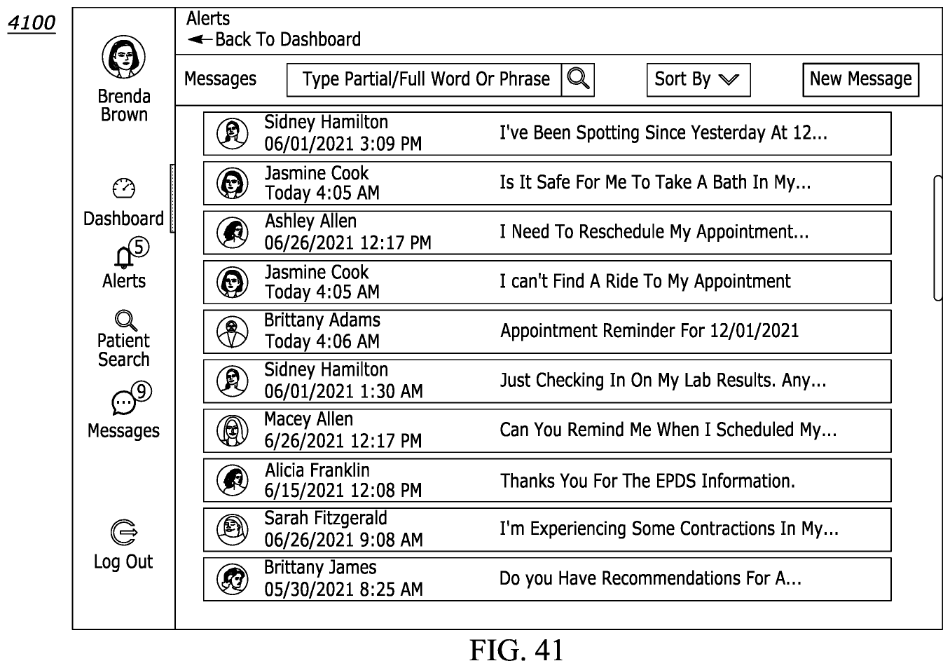

FIG. 40 illustrates an example display 4000 displaying search results of a search for a perinatal patient.

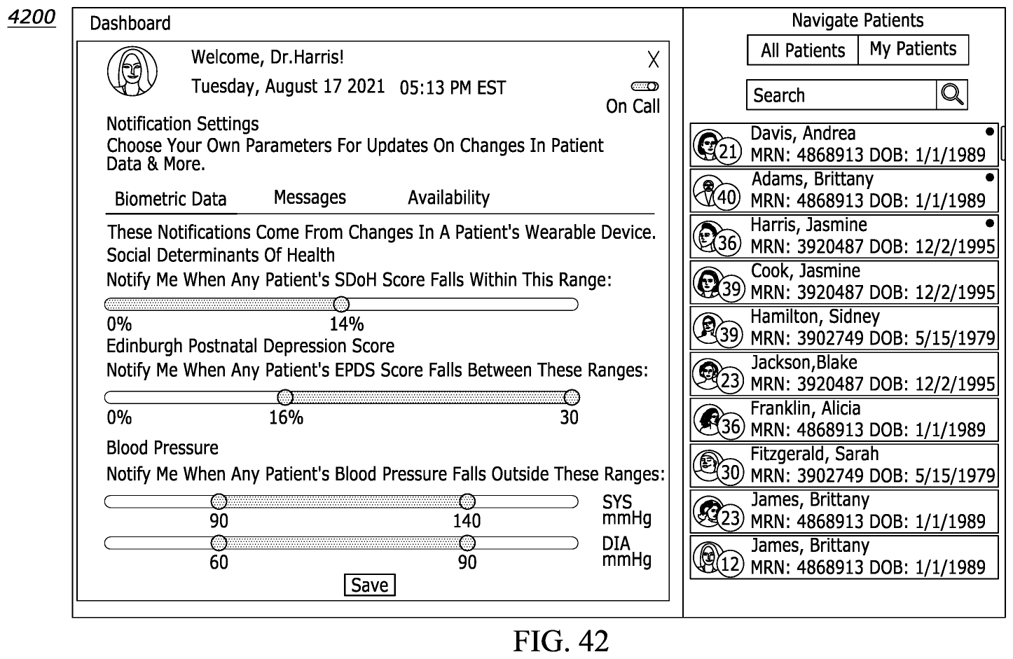

FIG. 41 illustrates an example display 4100 displaying a listing of text message conversations.

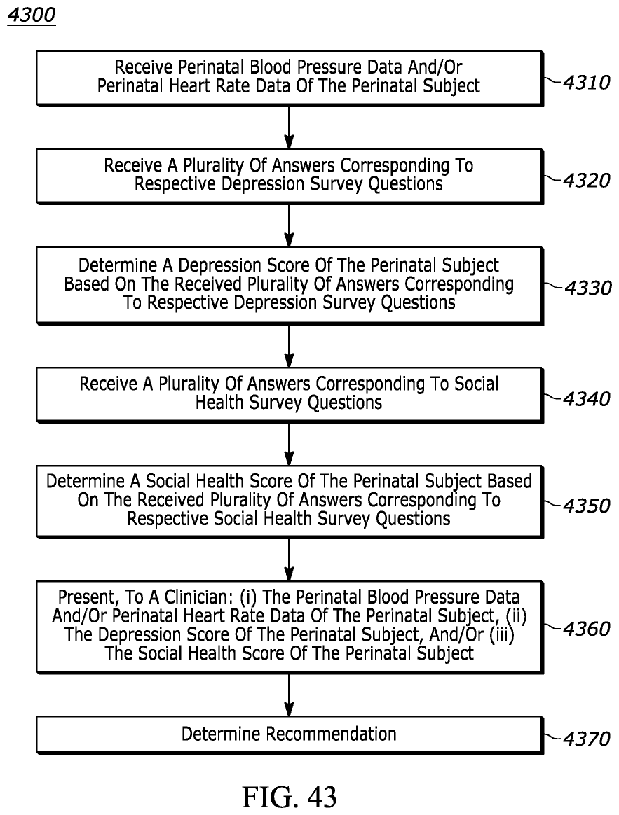

FIG. 42 illustrates an example display 4200 including sliderbars allowing a clinician to enter ranges for depression score, social health score, and blood pressure for which a notification (e.g., an alert) will be sent to the clinician. Although not illustrated in the example 4200, it should be understood that the clinician may also set such ranges (e.g., via sliderbar or any other suitable technique) for any of the other perinatal patient data (e.g., heartrate, blood oxygen data, respiration data, etc.).

### FURTHER EXAMPLE SYSTEMS & EXAMPLE METHODS

FIG. 44 illustrates an example of a portion of a perinatal monitoring and recommendation architecture 4400 that may be implemented as an example of the architecture 100 in FIG. 1. For example, the architecture 4400 may provide improved monitoring, treatment, and reporting healthcare services to prenatal patients and postpartum patients. In some embodiments, the healthcare services may be provided to a perinatal patient for a predetermined period of time following childbirth (e.g., for 6 months after childbirth, 1 year after child birth, 2 years after child birth, etc.). The architecture 4400 includes a healthcare computing device 4402 that may include one or more processors (not shown) such as one or more microprocessors, controllers, and/or any other suitable type of processor. The healthcare computing device 4402 may include and one or more memories (not shown) such as a volatile memory, non-volatile memory, etc. that are accessible by these processors, e.g., via a memory controller. The one or more memories may store instructions that, when executed by the one or more processors, cause the one or more processors to execute one or more application(s) discussed. Further, the healthcare computing device 4402 may include a network interface (not shown) for communicatively coupling the device 4402 to a network, such as a cloud-based network, extranet, intranet, Internet, etc. While such networks are not shown, the healthcare computing device 4402 will be understood as communicatively coupling with numerous different data sources and numerous different data collection/data reporting modalities, etc. In the illustrated example, measured patient data 4404 is communicated to the healthcare computing device 4402, for example, through a network (not shown). In the illustrated example, the measured patient data 4404 is presented by measured blood pressure data 4404a and measured heart rate data 4404b, for example, as may be captured in real time from one or more wearable devices coupled to the computing device 4402 through a network interface on the wearable device or through another computing device. It should be understood that the blood pressure and heart rate data may be any suitable measured data from which blood pressure and/or heart rate may be determined or other data correlative to either. Examples include resting heart rate, maximum heart rate, heart rate range, R-R interval (heart rate variability), VO₂, rating of perceived excursion, cardiac efficiency, systolic blood pressure, diastolic blood pressure, etc. Other sensor health data 4404c may also be received, such as weight.

As with other example architectures herein, measured patient data, such as the data 4400, may be provided to a medical data engine 4406 that may be configured to perform various different analyses on the received data. In various examples, the medical data engine 4406 performs data type detection, for example, by stripping and analyzing header fields or other data from which the engine can determine the type of data being received. In various examples, the medical data engine 4406 compares the values in the received data against previously determined threshold values or ranges of values to determine whether the values are in an alert range for the patient. These alert ranges may be patient specific, based on various factors associated with the patient, such as the patient's demographic data, or the patient's ranges in other categories (e.g., a patient's blood pressure measurement may result in a narrowed weight range before a threshold is reached, a patient's social determinants of health may result in a narrowed depression range before a threshold is reached, etc.) In various examples, the medical data engine 4406 compares the received data to historical measured data 4440 previously collected for the patient and stored in the healthcare computing device 4402, and does trendline determinations on the data, such as linear regressions, etc. to asses trends in the values.

Other patient health data received at the healthcare computing device 4402 include depression survey data 4408 that is received at a depression score engine 4410 and social determinants of health survey data 4412 that is received at a social health score engine 4414, respectively. In various examples, the depression score engine 4410 may be configured to receive raw depression data, such as patient input, caregiver input, or clinician input data obtained from answers corresponding to depression survey questions. These answers are stored as digital data and provided to the healthcare computing device 4402. In some examples, the depression score engine 4410 applies a depression scoring protocol to determine a depression score. In some examples, the engine 4410 uses different depression scoring protocols depending on whether the patient is prenatal or postpartum, where such different depression scoring protocols may be stored in protocol data stores 4416 and 4418, respectively. In some examples, the depression score is indicative of a current depression state. In other examples, the depression score may be predicted score for a future point in time, such as based on a linear regression generated by analyzing depressions scores over time. In some examples, the depression score includes an indication of whether the score is (or is predicted to be at a future point) in a predetermined, unsuitable value range.

In various examples, the social health score engine 4414 may be configured to receive raw social determinants of health data, such as patient input, caregiver input, or clinician input data obtained from answers corresponding to social determinants of health survey questions. These answers are stored as digital data and provided to the healthcare computing device 4402. The social health score engine 4414 applies a social determinants of health scoring protocol to determine a corresponding score. In some examples, the engine 4414 uses different social determinants of health scoring protocols depending on whether the patient is prenatal or postpartum, where such different social determinants of health scoring protocols may be stored in protocol data stores 4416 and 4418, respectively. In some examples, the social determinants of health score is indicative of a current state. In other examples, the social determinants of health score may be predicted score for a future point in time, such as based on a linear regression generated by analyzing social determinants of health scores over time. In some examples, the social determinants of health score includes an indication of whether the score is (or is predicted to be at a future point) in a predetermined, unsuitable value range.

In various examples, each of the engines 4410 and 4414 may be configured to perform scorings based on the received data 4408 and 4410, respectively, as well as demographic data 4420 on the patient. Such demographic data may include gender, age, marital status, ethnicity, race, national origin, language, address, education level, occupation, social history (smoking, alcohol consumption, drug use, exercise, diet), etc. That is, in some examples, the depression scores and social health scores generated by the healthcare computing device 4402 may be based on multiple different types of data provided to the engines 4410 and 4414, respectively.

In some examples, the healthcare computing device 4402 is configured to generate a multifactor scoring for a patient, by for example combining output data from the medical data engine 4406, the depression score engine 4410, and the social health score engine 4414. In an example, medical data such as blood pressure, heart rate data, and/or other sensor data, along with a depression score, and a social health score are provided to a machine learning engine 4422 trained to generate an overall multifactor health score for the patient. That multifactor health score may then be used to adjust patient monitoring, patient health alerts, and/or patient health reporting. In some examples, that multifactor health score may be used by a healthcare provider to inform treatment options for the patient.

In some examples, the data 4404, 4408, 4412, and/or 4420 may be communicated directly to the trained machine learning engine 4422 for generating a multifactor health score. In some examples, the machine learning engine 4422 is trained to generate both a depression score and a social determinants of health score, for example, by containing different machine learning layers or sub-models each trained to generate the respective scores. In some examples, the machine learning engine 422 is trained to generate a multifactor health score directly from the received data, without needing to determine depression scores or social determinants of health scores.

In some examples, the machine learning engine 4422 may be trained using large training datasets 4424 (100+, 1000+, or 10000+ entries) of patient data containing, for each patient, at least one medical data, depression survey score data, and social determinants of health survey data. In some examples, where not all such data is available for patients in the training datasets, an inference engine may be used to generate synthetic data to complete the patient data entries. The training datasets may further include physician data 4426, e.g., indicating a treatment provided to the patient or a change in medical data, depression, and or social health monitoring for the patient.

Example architectures for the machine learning engine 4422 include a logistic regression based model trained to predict a binary outcome, such as whether a patient is in need of treatment, a patient is in need of increased monitoring, a patient is in in need of a change in monitoring, etc. Other example machine learning architectures include support vector machines (SVMs) which can predict both binary and continuous outcomes, such as to predict the risk of patient mortality. Yet other machine learning architectures include random forests models, deep learning models formed of neural networks, gradient boosting models, Nave Bayes models, or other machine learning algorithms (MLA).

The healthcare computing device 4402 further includes a decision engine/report generator 4450 that receives output data from the medical data engine 4406, depression scores from the engine 4410, and social determinants of health scores from the engine 4414. In some examples, with the machine learning engine 4422 trained to generate a multifactor health score based on inputs from all three engines 4406, 4410, and 4414, the engine/generator 4450 receives output data from machine learning engine 4422.

In the illustrated example, the decision engine/report generator 4450 includes a diagnosis engine 4452, a clinician alert engine 4454, a messaging engine 4456, a recommendation engine 4458, a GUI engine 4460, and a report generator engine 4462.

In various examples, the diagnosis engine 4452 is configured to compare the output data from the engines 4406, 4410, and 4414 or from the machine learning engine 4422 to diagnosis data for a sample patient population to determine if the output data indicates that the patient is experiencing a potential morbidity event, advanced depression requiring treatment, poor social health requiring treatment, blood pressure (BP) values specific to the patient that require further treatment, heart rate (HR) values specific to the patient that require further treatment, weight values specific to the patient that require further treatment, or some combination of the above that require further treatment.

In various examples, the clinician alert engine 4454 is configured to assess the output data from the engines 4406, 4410, and 4414 or from the machine learning engine 4422 and compare against alert threshold values, against historical data from the patient for determine trendlines, or a combination of both, to determine if an electronic alert should be generated and communicated to the clinician associated with the patient. In some examples, the clinician alert engine communicates determined alerts to other engines, such as the GUI engine 4460 configured to generate one or more of the GUIs described herein, the messaging engine 4456 configured to generate automate messages from a data store of available messages with instructions to the patient, the recommendation engine 4458 configured to generate instructions to modify survey questions such as the depression survey questions, social determinants of health survey questions, and/or quality of life survey questions, and the report generator engine 4462 configured to generate and store electronic reports of determined data for the patient.

FIG. 43 shows an example method 4300 of monitoring a perinatal patient. Although the blocks of example method 4300 are discussed below as being performed by the one or more processors 120 of the healthcare computing device 102, it should be understood that any or all of the blocks may be performed by any other suitable component(s), such as one or more processors of a clinician computing device 175, and/or one or more processors of a patient computing device 165.

The example method 4300 may begin at block 4310 where the one or more processors 120 receive perinatal blood pressure data and/or perinatal heart rate data of the perinatal patient. In some examples, the perinatal blood pressure data and/or perinatal heart rate data is received from the perinatal patient computing device 165 and/or the electronic medical device 162. In some embodiments, the perinatal blood pressure data and/or perinatal heart rate data include time stamps indicating when the blood pressure data and/or heart rate data was measured.

At block 4320, the one or more processors 120 may receive (e.g., from the perinatal patient computing device 165, or from any other suitable device) a plurality of answers corresponding to respective depression survey questions. The perinatal patient may have entered the answers via any suitable technique. For example, the perinatal patient may have entered answers: as numerical values; via a slider bar(s), as yes/no answers, etc.

At block 4330, the one or more processors 120 may determine a depression score of the perinatal patient based on the received plurality of answers corresponding to respective depression survey questions.

At block 4340, the one or more processors 120 receive (e.g., from the perinatal patient computing device 165, or from any other suitable device) a plurality of answers corresponding to social health survey questions. The perinatal patient may have entered the answers via any suitable technique. For example, the perinatal patient may have entered answers: as numerical values; via a slider bar(s), as yes/no answers, etc.

At block 4350, the one or more processors 120 determine a social health score of the perinatal patient based on the received plurality of answers corresponding to respective social health survey questions. In some examples, the social health score is a social determinants of health (SDoH) score. In some examples, the one or more processors 120 also determine subscores of the social health score.

At block 4360, the one or more processors present, to a clinician: (i) the perinatal blood pressure data and/or perinatal heart rate data of the perinatal patient, (ii) the depression score of the perinatal patient, and/or (iii) the social health score of the perinatal patient. In some embodiments, the presentation is made via the clinician computing device 175.

In some examples, the social health score is presented as a numerical value, and the subscores are presented in graphical form, as in the example of FIG. 6 (e.g., the social health score is presented as a numerical score of 5, and the subscores are presented graphically as curved bars).

At block 4370, the one or more processors may determine a recommendation for the perinatal patient based on (i) the perinatal blood pressure data and/or perinatal heart rate data of the perinatal patient, (ii) the depression score of the perinatal patient, and/or (iii) the social health score of the perinatal patient. The recommendation may be presented to the clinician.

In some examples, the recommendation comprises a recommended treatment or a recommendation for an appointment with a clinician. In some such examples, clinician is a physician or a social worker.

In some examples, the recommendation is first presented to the clinician; and, upon approval and/or modification by the clinician, the recommendation is forwarded to the perinatal patient.

In some examples, the recommendation includes a recommended timeframe to complete the recommendation. In some examples, the recommendation is a recommendation to change a periodicity of a healthcare appointment (e.g., increase visits with a social worker from once a month to once a week, etc.).

In some examples, the determination of the recommendation for the patient may be based on correlations between any of: (i) time stamps indicating when the perinatal blood pressure data and/or perinatal heart rate data was measured, (ii) time stamps indicating when the perinatal patient answered the depression survey questions, (iii) time stamps indicating when the perinatal patient answered the social health survey questions, and/or (iv) time stamps indicating when the perinatal patient answered the quality of life survey questions.

In some examples, the determination of the recommendation comprises routing, to a trained machine learning algorithm: (i) the perinatal blood pressure data and/or perinatal heart rate data of the perinatal patient, (ii) the depression score of the perinatal patient, and/or (iii) the social health score of the perinatal patient.

The machine learning algorithm may be trained via any suitable technique. For example, the machine learning algorithm may be trained to determine recommended treatments by routing historical data into the machine learning algorithm. Examples of the historical data include historical: (i) perinatal blood pressure data and/or perinatal heart rate data of patients, (ii) depression scores of patients, (iii) social health scores of patients, (iv) treatments of patients, and/or (v) outcomes of treatments of the patients. In some such embodiments, the machine learning algorithm may be trained using the above (i)-(iii) as inputs to the machine learning model (e.g., also referred to as independent variables, or explanatory variables), and the above (iv)-(v) used as the outputs of the machine learning model (e.g., also referred to as a dependent variables, or response variables). Put another way, each of the above (i)-(iii) may have an impact on (iv)-(v), which the machine learning algorithm is trained to find.

In some embodiments, the machine learning algorithm may be trained on a subset of the historical data corresponding to a particular racial group, thereby improving the accuracy of the machine learning algorithm for that particular racial group.

In some embodiments, the treatment also includes a periodicity of the treatment. For example, the recommendation may be that the perinatal patient see a social worker at a certain periodicity (e.g., once a month, twice a month, etc.).

Some further embodiments also leverage combinations of postpartum data and prenatal data. For example, the one or more processors 120 may receive prenatal blood pressure data and/or prenatal heart rate data of the perinatal patient; prenatal answers corresponding to respective depression survey questions; prenatal answers corresponding to social health survey questions, and/or prenatal answers corresponding to quality of life survey questions. And, the one or more processors 120 may also receive postpartum blood pressure data and/or postpartum heart rate data of the perinatal patient; postpartum answers corresponding to respective depression survey questions; postpartum answers corresponding to social health survey questions; and/or postpartum answers corresponding to quality of life survey questions. Any or all of the prenatal data and/or postpartum data may be presented to the clinician. In some examples, the prenatal data may be used to create a baseline for the perinatal patient to assist in making the recommendations for the patient.

In some examples, a machine learning algorithm is trained to determine the recommendations by routing historical data into the machine learning algorithm. In some such examples, historical data comprises historical: (i) prenatal and/or postpartum blood pressure data and/or prenatal and/or postpartum heart rate data of patients, (ii) prenatal and/or postpartum depression scores of patients, (iii) prenatal and/or postpartum social health scores of patients, (iv) treatments of patients, and/or (v) outcomes of treatments of the patients. In some such embodiments, the machine learning algorithm may be trained using the above (i)-(iii) as inputs to the machine learning model (e.g., also referred to as independent variables, or explanatory variables), and the above (iv)-(v) used as the outputs of the machine learning model (e.g., also referred to as a dependent variables, or response variables). Put another way, each of the above (i)-(iii) may have an impact on (iv)-(v), which the machine learning algorithm is trained to find.

In some embodiments, the machine learning algorithm may be trained on a subset of the historical data corresponding to a particular racial group, thereby improving the accuracy of the machine learning algorithm for that particular racial group.

In some embodiments, the treatment also includes a periodicity of the treatment. For example, the recommendation may be that the perinatal patient see a social worker at a certain periodicity (e.g., once a month, twice a month, etc.). In examples where the treatment comprises medication treatment, a periodicity and/or dosage of the medication treatment may also be included in the recommendation.

In some further examples, if the depression score is below a depression score threshold value, the one or more processors 120 alert the clinician.

In some embodiments, if the social health score is below a social health score threshold value, the one or more processors 120 alert the clinician.

In some embodiments, the one or more processors 120 determine a combined risk score based on two or more of: (i) the perinatal blood pressure data and/or perinatal heart rate data of the perinatal patient, (ii) the depression score, and/or (iii) the social health score. The one or more processors 120 may further trigger an alert to the clinician based on a comparison between the combined risk score and a combined risk score threshold value (e.g., the trigger occurs on the combined risk score being above a predetermined combined risk score threshold value).

Further regarding the example flowchart provided above, it should be noted that all blocks are not necessarily required to be performed. Moreover, additional blocks may be performed although they are not specifically illustrated in the example flowchart.

### Other Matters

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Furthermore, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

1. A computer-implemented method for monitoring a perinatal patient from diagnosis through twelve months postpartum to a childbirth event via real time patient data and alerts, the method comprising:
retrieving, via one or more processors, blood pressure data and/or heart rate data of a patient from one or more electronic medical devices;
receiving, via the one or more processors, from a patient user device corresponding to the patient, a plurality of answers corresponding to respective depression survey questions;
determining, via the one or more processors, a depression score of the patient based on the received plurality of answers corresponding to respective depression survey questions;
receiving, via the one or more processors, from the patient user device, a plurality of answers corresponding to social determinants of health survey questions;
determining, via the one or more processors, a social determinants of health score of the patient based on the received plurality of answers corresponding to respective social determinants of health survey questions;
presenting, via the one or more processors, to a clinician: (i) the blood pressure data and/or heart rate data of the patient, (ii) the depression score of the patient, and (iii) the social determinants of health score of the patient.

2. The computer-implemented method of claim 1, wherein the blood pressure data and/or heart rate data is measured via a Micro-Electro-Mechanical Systems (MEMS) sensor.

3. The computer-implemented method of claim 1, wherein the blood pressure data and/or heart rate data is measured via a pressure sensor, and not via an optical sensor, thereby improving blood pressure data quality and/or heart rate data quality for patients with dark skin tone.

4. The computer-implemented method of claim 1, wherein the blood pressure data and/or heart rate data comprises systolic blood pressure data, and diastolic blood pressure data.

5. The computer-implemented method of claim 1, wherein the blood pressure data and/or heart rate data include time stamps indicating when the blood pressure data and/or heart rate data was measured.

6. The computer-implemented method of claim 1, further comprising:
determining, via the one or more processors, a recommendation for the patient based on (i) the blood pressure data and/or heart rate data of the patient, (ii) the depression score of the patient, and/or (iii) the social determinants of health score of the patient; and
presenting, via the one or more processors, the recommendation to the clinician.

7. The computer-implemented method of claim 6, wherein the recommendation comprises a recommended treatment or a recommendation for an appointment with a clinician.

8. The computer-implemented method of claim 7, wherein the clinician is a physician or a social worker.

9. The computer-implemented method of claim 6, wherein the recommendation includes a recommended timeframe to complete the recommendation.

10. The computer-implemented method of claim 6, wherein:
the blood pressure data and/or heart rate data include time stamps indicating when the blood pressure data and/or heart rate data was measured;
the received plurality of answers corresponding to respective depression survey questions include time stamps indicating when the patient answered the depression survey questions;
the received plurality of answers corresponding to respective social determinants of health survey questions include time stamps indicating when the patient answered the social health survey questions; and
the determination of the recommendation for the patient is further based on correlations between any of: (i) the time stamps indicating when the blood pressure data and/or heart rate data was measured, (ii) the time stamps indicating when the patient answered the depression survey questions, and/or (iii) the time stamps indicating when the patient answered the social determinants of health survey questions.

11. The computer-implemented method of claim 6, wherein the determining the recommendation comprises routing, to a trained machine learning algorithm: (i) the blood pressure data and/or heart rate data of the patient, (ii) the depression score of the patient, and/or (iii) the social determinants of health score of the patient.

12. The computer-implemented method of claim 1, further comprising:
training, via the one or more processors, a machine learning algorithm to determine recommended treatments by routing historical data into the machine learning algorithm; and
wherein the historical data comprises historical: (i) blood pressure data and/or heart rate data of patients, (ii) depression scores of patients, (iii) social health scores of patients, (iv) treatments of patients, and/or (v) outcomes of treatments of the patients.

13. The computer-implemented method of claim 1, further comprising:
receiving, via the one or more processors, historical data;
determining, via the one or more processors, a subset of the historical data corresponding to a particular racial group; and
training, via the one or more processors, a machine learning algorithm to determine recommendations by routing the subset of historical data into the machine learning algorithm; and
wherein the historical data comprises historical: (i) blood pressure data and/or heart rate data of patients, (ii) depression scores of patients, (iii) social health scores of patients, (iv) treatments of patients, and/or (v) outcomes of treatments of the patients.

14. The computer-implemented method of claim 1, wherein:
the patient is a prenatal patient;
the blood pressure data and/or heart rate data is prenatal blood pressure and/or prenatal heartrate data;
the depression score is a prenatal depression score; and
the social determinants of health score is a prenatal determinants of social health score.

15. The computer-implemented method of claim 1, further comprising determining, via the one or more processors, subscores of the social determinants of health score; and
wherein the presenting comprises presenting, via the one or more processors, the social determinants of health score as a numerical value, and presenting, via the one or more processors, the subscores in graphical form.

16. The computer-implemented method of claim 1, wherein:
the patient is a postpartum patient;
the blood pressure data and/or heart rate data is postpartum blood pressure and/or postpartum heartrate data;
the depression score is a postpartum depression score;
the social determinants of health score is a postpartum determinants of social health score; and
the method further comprises:
receiving, via one or more processors, prenatal blood pressure data and/or prenatal heart rate data of the postpartum patient;
receiving, via the one or more processors, from the patient user device corresponding to the postpartum patient, a plurality of prenatal answers corresponding to respective depression survey questions;
determining, via the one or more processors, a prenatal depression score of the postpartum patient based on the received plurality of prenatal answers corresponding to respective depression survey questions;
receiving, via the one or more processors, from the patient user device, a plurality of prenatal answers corresponding to social determinants of health survey questions;
determining, via the one or more processors, a prenatal social determinants of health score of the postpartum patient based on the received plurality of prenatal answers corresponding to respective social determinants of health survey questions; and
presenting, via the one or more processors, to a clinician: (i) the prenatal blood pressure data and/or prenatal heart rate data of the postpartum patient, (ii) the prenatal depression score of the postpartum patient, and (iii) the prenatal social determinants of health score of the postpartum patient.

17. The computer-implemented method of claim 16, further comprising:
determining, via the one or more processors, a recommendation for the postpartum patient based on (i) the postpartum blood pressure data and/or postpartum heart rate data of the postpartum patient, (ii) the depression score of the postpartum patient, (iii) the social determinants of health score of the postpartum patient, (iv) the prenatal blood pressure data and/or prenatal heart rate data of the postpartum patient, (v) the prenatal depression score of the postpartum patient, and/or (vi) prenatal the social determinants of health score of the postpartum patient; and
presenting, via the one or more processors, the recommendation to the clinician.

18. The computer-implemented method of claim 1, further comprising:
training, via the one or more processors, a machine learning algorithm to determine recommendations by routing historical data into the machine learning algorithm; and
wherein the historical data comprises historical: (i) prenatal and/or postpartum blood pressure data and/or prenatal and/or postpartum heart rate data of patients, (ii) prenatal and/or postpartum depression scores of patients, (iii) prenatal and/or postpartum social health scores of patients, (iv) treatments of patients, and/or (v) outcomes of treatments of the patients.

19. The computer-implemented method of claim 1, further comprising:
if the depression score is below a depression score threshold value, alerting, via the one or more processors, the clinician.

20. The computer-implemented method of claim 1, further comprising:
if the social determinants of health score is below a social determinants of health score threshold value, alerting, via the one or more processors, the clinician.

21.-35. (canceled)
